(19) United States
(12) Patent Application Publication
(10) Pub. No.: US 2024/0415383 A1
(43) Pub. Date: Dec. 19, 2024

(54) SYSTEMS AND METHODS FOR RETINAL SPECTRAL IMAGING CALIBRATION PHANTOM

(71) Applicant: RETISPEC INC., Toronto (CA)

(72) Inventors: Eliav Shaked, Toronto (CA); Alon Hazan, Toronto (CA); Joni Petteri Teikari, Toronto (CA); Tommaso Alterini, Toronto (CA)

(73) Assignee: RETISPEC INC., Toronto (CA)

(21) Appl. No.: 18/806,795

(22) Filed: Aug. 16, 2024

Related U.S. Application Data
(63) Continuation of application No. PCT/IB2023/000388, filed on Jun. 29, 2023.
(60) Provisional application No. 63/375,726, filed on Sep. 15, 2022.
(60) Provisional application No. 63/356,652, filed on Jun. 29, 2022.

(51) Int. Cl. A61B 3/00

(52) U.S. Cl. CPC A61B 3/0025

(57) ABSTRACT

A phantom eye includes a curved reflectance standard having a light-receiving surface configured to be illuminated by light and one or more ocular media components positioned such that the light passes through the one or more ocular media components prior to illuminating the light-receiving surface of the curved reflectance standard. The curved reflectance standard mimics an optical property of a retina of a biological eye. The one or more ocular media components mimic an optical property of the biological eye.

### CROSS-REFERENCE TO RELATED APPLICATIONS

This application is a bypass continuation patent application of PCT/IB2023/000388, filed Jun. 29, 2023, which claims the benefit of and priority to U.S. Provisional Patent Application No. 63/356,652, filed Jun. 29, 2022, and U.S. Provisional Patent Application No. 63/375,726, filed Sep. 15, 2022, the contents of which are incorporated herein by reference in their entirety.

### FIELD

This disclosure relates to systems and methods for calibrating retinal spectral imaging systems.

### BACKGROUND

Alzheimer's disease (AD) is a debilitating and fatal neurodegenerative disease. Confirmation of the disease is commonly performed post-mortem. Some existing conventional systems for diagnosis involve either highly invasive procedures or imaging devices that are often inaccessible or inappropriate due to cost, complexity, or the use of harmful radioactive tracers.

There is a need for a non-invasive detection system that is easily operable and accessible by clinicians for screening patient populations for early detection of AD-associated pathologies, diagnosis, and tracking of patient response to preventative or treatment interventions.

The optic nerve and retina are developmental outgrowths of the brain and many conditions affecting the brain manifest in these structures as well, such as amyloid beta (Aβ) protein accumulation, changes to the structure of retinal layers, and other changes in chemical composition, structure, and function. The eye is easily examined using a variety of non-invasive light-based techniques to identify these physical changes, making optical analysis highly suited for the present needs. For instance, spectral imaging, which makes use of an imaging sensor (such as a charge-coupled device (CCD) or complementary metal oxide semiconductor (CMOS) sensor) that can resolve and measure multiple wavelengths of light arriving from each spatial element across a two-dimensional object or scene, may be used to examine an eye. Improved systems and methods for calibrating retinal spectral imaging systems for imaging an eye are desired.

### SUMMARY

The present disclosure relates to a phantom eye, including a curved reflectance standard having a light-receiving surface configured to be illuminated by light and one or more ocular media components positioned such that the light passes through the one or more ocular media components prior to illuminating the light-receiving surface of the curved reflectance standard. The curved reflectance standard mimics an optical property of a retina of a biological eye. The one or more ocular media components mimic an optical property of the biological eye.

The present disclosure relates to a system including a phantom eye including a curved reflectance standard having a light-receiving surface configured to be illuminated by light and one or more ocular media components positioned such that the light passes through the one or more ocular media components prior to illuminating the light-receiving surface of the curved reflectance standard, wherein. The curved reflectance standard mimics an optical property of a retina of a biological eye, and the one or more ocular media components mimic an optical property of the biological eye. The system also includes a light source configured to emit light to the curved reflectance standard and a sensor configured to detect light reflected by the curved reflectance standard.

The present disclosure relates to a method including imaging a phantom eye with alight source of a lighting assembly to generate a reference image, where the phantom eye includes a curved reflectance standard having a light-receiving surface configured to be illuminated by light and one or more ocular media components positioned between the curved reflectance standard and the lighting assembly, where the curved reflectance standard mimics an optical property of a retina of a biological eye, and the one or more ocular media components mimic an optical property of the biological eye. The method further includes imaging the biological eye with the light source of the lighting assembly to generate a spectral image of the biological eye, and adjusting the spectral image of the biological eye based at least in part on the reference image.

The present disclosure relates to a system, including a phantom eye, alighting assembly including a light source configured to direct light to the phantom eye and a sensor configured to detect light reflected from the phantom eye, and a processor in communication with the lighting assembly and programmed to correct a spectral retinal image with a spectral calibration image of the phantom eye.

The present disclosure relates to an apparatus including at least one processor and at least one storage medium having encoded thereon executable instructions that, when executed by the at least one processor, cause the at least one processor to carry out a method. The method includes adjusting a hyperspectral image of a biological eye based at least in part on a reference image captured using a phantom eye, where the phantom eye includes a curved reflectance standard having a light-receiving surface configured to be illuminated by light and one or more ocular media components positioned such that the light passes through the one or more ocular media components prior to illuminating the light-receiving surface of the curved reflectance standard.

The present disclosure relates to a method of using a phantom eye, including imaging a phantom eye with a light source of a lighting assembly to generate a reference image, where the reference image is configured to be used to calibrate an imaging system. The phantom eye includes a curved reflectance standard having a light-receiving surface configured to be illuminated by light and one or more ocular media components positioned between the curved reflectance standard and the lighting assembly, where the curved reflectance standard mimics an optical property of a retina of a biological eye and the one or more ocular media components mimic an optical property of the biological eye.

The present disclosure relates to at least one non-transitory computer-readable storage medium having encoded thereon executable instructions that, when executed by at least one processor, cause the at least one processor to carry out a method including adjusting a hyperspectral image of a biological eye based at least in part on a reference image captured using a phantom eye. The phantom eye includes a curved reflectance standard having a light-receiving surface configured to be illuminated by light and one or more ocular media components positioned such that the light passes through the one or more ocular media components prior to illuminating the light-receiving surface of the curved reflectance standard.

### BRIEF DESCRIPTION OF DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A shows a schematic of an exemplary embodiment of a spectral imaging system;

FIG. 1B shows a schematic of an exemplary embodiment of a spectral calibration system;

FIG. 1C shows an ophthalmic imaging system for reforming a field curvature corrected image on an imaging sensor;

FIG. 2 shows an exemplary embodiment of a spectral calibration system including a phantom eye;

FIG. 3, shows a detailed cross-sectional view of the phantom eye of FIG. 2;

FIG. 4, shows an exemplary embodiment of a spectral calibration system including a phantom eye;

FIG. 5, shows an exemplary embodiment of a spectral calibration system including a phantom eye;

FIG. 6, shows an exemplary embodiment of a spectral calibration system including a phantom eye;

FIG. 7, shows an exemplary embodiment of a spectral calibration system including a phantom eye and a spectral filter;

FIG. 8, shows a cross-sectional view of a phantom eye;

FIG. 9A shows an exemplary embodiment of a spectral calibration system having a phantom eye and a track;

FIG. 9B shows an exemplary embodiment of a spectral calibration system having a phantom eye and a rotary wheel;

FIG. 9C shows a front view of the rotary wheel of FIG. 9B;

FIG. 9D shows an exemplary embodiment of a spectral calibration system having a phantom eye including internal slots;

FIG. 10 shows an exemplary embodiment of a spectral calibration system including a phantom eye and a beam splitter;

FIG. 11 shows exemplary embodiments of phantom eyes of various curvatures;

FIG. 12 shows an exemplary embodiment of a spectral calibration system including a phantom eye of an integrating sphere;

FIG. 13 shows an exemplary embodiment of a spectral calibration system including a phantom eye of an integrating sphere;

FIG. 14 shows an exemplary embodiment of a spectral calibration system including an electronic control unit;

FIG. 15A shows a block diagram of illustrative internal components of the electronic control unit of FIG. 14;

FIG. 15B shows a block diagram of illustrative logic modules contained within a memory component of the electronic control unit of FIG. 15A

FIG. 16 shows an illustrative control network;

FIG. 17 shows a flow diagram of an illustrative method of adjusting a spectral image of a biological eye with a spectral image of a phantom eye;

FIG. 18 shows normalized spectral curves of a fovea image from a hyperopic human eye;

FIG. 19 shows magnified spectral curves of FIG. 18;

FIG. 20 shows cross sections of retinal hyperspectral datacubes normalized with reflectance standards; and

FIG. 21 shows spectral curves of retinal hyperspectral datacubes normalized with flat and curved reflectance standards.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments. Embodiment examples are described as follows with reference to the figures. Identical, similar, or identically acting elements in the various figures are identified with identical reference numbers and a repeated description of these elements is omitted in part to avoid redundancies. “Anterior,” as used herein, refers to the direction away from the curved reflectance standard and closer to a light source configured to illuminate a light-receiving surface of the curved reflectance standard. For example, light from the light source passes through a component anterior to the curved reflectance standard before illuminating the light-receiving surface of the curved reflectance standard. Therefore, as an example, if a first component is positioned anterior the curved reflectance standard, and a second component is positioned anterior the first component, light from the light source passes through the second component before the first component and passes through the first component before illuminating the light-receiving surface of the curved reflectance standard. “Posterior,” as used herein, refers to the direction opposite of the anterior direction.

The instant systems and methods can be used to calibrate spectral imaging systems that may be used for detecting the existence of one or more Alzheimer's disease (AD) associated pathologies or pathologies associated with other neurogenerative diseases, such as, for example, Parkinson's disease (PD), amyotrophic lateral sclerosis (ALS), multiple sclerosis, Prion disease, Motor neurone diseases (MND), Huntington's disease (HD), Spinocerebellar ataxia (SCA), Spinal muscular atrophy (SMA), cerebral amyloid angiopathy (CAA), other forms of dementia, and similar diseases of the brain or the nervous system. In some embodiments, the spectral imaging systems and method of the present disclosure may detect biomarkers indicative of tau pathologies or tauopathies, including, without limitation, total (T-tau), Tau PET, and phosphorylated tau (P-tau). In some embodiments, the biomarkers indicative of a Tauopathy include, but are not limited to, phosphorylated paired helical filament tau (pTau), Early Tau phosphorylation, Late Tau phosphorylation, pTau181, pTau217, pTau231, total Tau, Plasma AB 42/40, Neurofibrillary tangles (NFTs) and aggregation of misfolded tau protein. In some embodiments, neurofilament light protein (NFL), neurofilaments (NFs) or abnormal/elevated neurofilament light protein (NFL) concentration can be detected. In some embodiments, surrogate markers of a neurodegenerative disorder or neuronal injury can be detected, for example, retinal and optic nerve volume loss or other changes, degeneration within the neurosensory retina, and optic disc axonal injury. In some embodiments, an inflammatory response or neuroinflammation may be detected and may be indicative of neurogenerative disease. In some embodiments, such inflammatory response may be detected in the retinal tissue. Examples of such responses include, but are not limited to, retinal microglia activation, degenerating ganglion cells (ganglion neuron degeneration) or astrocyte activation. Other protein aggregates or biomarkers useful in the imaging methods and systems of the present disclosure include alpha synuclein and TDP43 (TAR DNA binding protein-43) and others described, for example, in Biomarkers for tau pathology (Molecular and Cellular Neuroscience, Volume 97, June 2019, Pages 18-33), incorporated herein by reference in its entirety. In some embodiments, the imaging systems and methods of the present disclosure can be used to detect the presence or absence of protein aggregates or biomarkers indicative of one or more neurogenerative diseases in the patient's eye tissue, brain tissue, tissues of the central nervous system, peripheral nervous system, or in the cerebrospinal fluid (CSF) or any other tissue where such formations or their biomarkers occur. In some embodiments, the imaging systems and methods of the present disclosure detect protein aggregates or biomarkers indicative of one or more neurogenerative diseases without using a dye or ligand. It should be appreciated that the foregoing are non-limiting examples, and the spectral imaging systems described herein may be used to detect any number of desirable biomarkers or diseases.

Calibrating spectral imaging systems, as discussed herein, may include correcting spectral images, generated of a biological eye with a spectral imaging system, of one or more aberrations or artifacts. During spectral imaging, the reflected light arriving at the imaging sensor changes as a function of absorbance, transmittance, scattering, reflectance, and emission of the object, or scene, that is imaged and the properties of the illumination light source, as well.

“Spectral imaging,” as used herein, may relate to imaging of an object at one or more bands of any wavelengths across the electromagnetic spectrum. For example, “spectral imaging,” may refer to hyperspectral imaging or multispectral imaging. In some embodiments, the spectral imaging can be performed by a monochromatic sensor, RGB sensor, hyperspectral sensor, multispectral sensor, polarimetric, or a spectropolarimetric sensor.

Because flat white light (light that has the same intensity at every wavelength) illumination sources are not generally available or practical, it is a common practice to measure the spectral variation (the change in intensity as a function of wavelength) of the light source, itself after reflecting on an object or scene. In turn, the spectral variation may be used as a calibration measurement to correct the measured image of the object or scene for the light source, sensor, and optical system properties, retaining only the spectral intensity variations introduced by the object or scene under examination. This calibration reference measurement is generally obtained by placing a white reflective target (a target that is spectrally flat and reflects all wavelengths of light with the same efficiency), or reflectance standard, at the optical plane at which would be located an object that will be examined, illuminating the white reflective target with the light source, and measuring the light reflected by the white reflective target with the sensor. The measurement of light reflected by the white reflective target is used as the reference that characterizes the spectral properties of the light source, the efficiency of the sensor, the transmittance of the optical system, and the spectral radiance in wavelength that would be received during imaging of an object under examination. When imaging the object under examination, the spectral measurement for each spatial element can then be corrected using the reference. For example, the measured intensity of light reflected by the object under examination at each wavelength may be divided by the reference intensity measured at the same wavelength when imaging the reflective target.

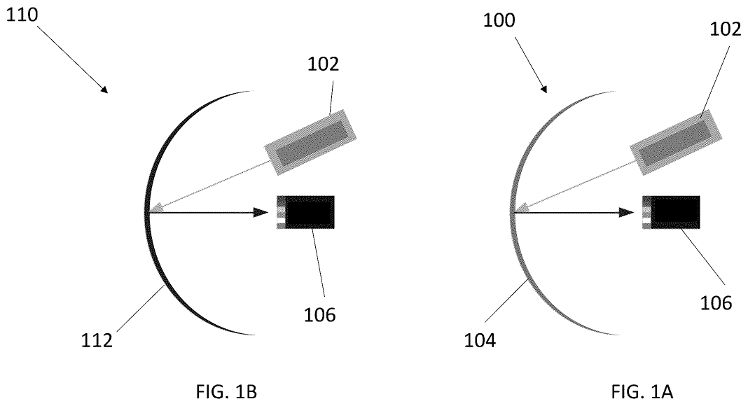

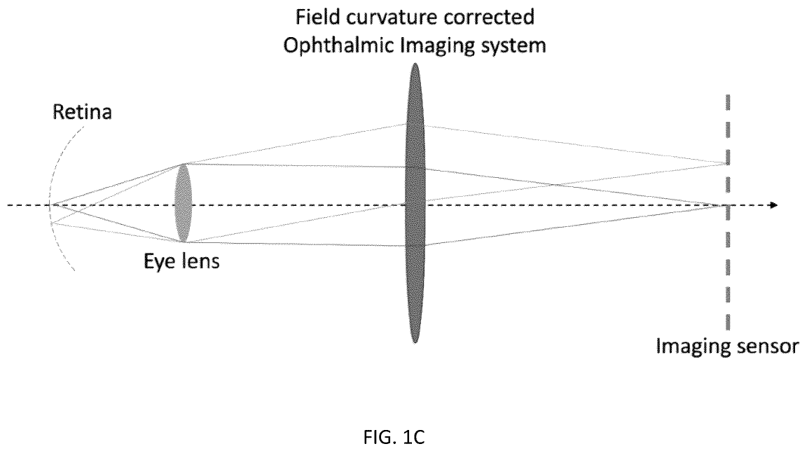

With reference to FIG. 1A, a basic spectral imaging system 100 is depicted, including a light source 102, an object 104 under examination, and a sensor 106. A mathematical relationship between the intensity of light emitted from the light source 102 (and received by the object 104 under examination), the reflectance of the object 104 under examination, and the intensity of light measured by the sensor 106 (and reflected by the object 104 under examination) is as follows, where R_{object}(λ) represents the reflectance of the object 104, S_{object}(λ) represents the intensity of light measured by the sensor 106, and I(λ) represents the intensity of light emitted by the light source 102.

$${R}_{object} ( λ ) = \frac{{S}_{object} ( λ )}{I ( λ )}$$

Referring to FIG. 1B, a basic spectral calibration system 110 is depicted, including a reflectance standard 112 in place of the object 104, the light source 102, and the sensor 106. A mathematical relationship between the intensity of light emitted from the light source 102 (and received by the reflectance standard 112), the reflectance of the reflectance standard 112, and the intensity of light measured by the sensor 106 (and reflected by the reflectance standard 112) is as follows, where R_{standard}(λ) represents the reflectance of the reflectance standard 112, S_{standard}(λ) represents the intensity of light measured by the sensor 106, and I(λ) represents the intensity of light emitted by the light source 102.

$${R}_{standard} ( λ ) = \frac{{S}_{standard} ( λ )}{I ( λ )}$$

Based on the above relationships, the intensity of light measured by the sensor 106 and reflected by the object 104 under examination can be corrected for variations in the intensity of the light source 102 with the below equation, where R_{corrected}(λ) represents the reflectance of the object 104 corrected for variations in the intensity of the light source 102, the sensor 106, and the imaging system 100.

$${R}_{corrected} ( λ ) = \frac{{S}_{object} ( λ )}{{S}_{standard} ( λ )}$$

The accuracy of such corrections and calibration methods may be limited by the design and placement of the reflectance standard 112, however. For instance, for accurate calibration, the reflectance standard 112 should be positioned in the same optical plane, relative the light source 102 and sensor 106, as the object 104 under examination and imaged under the exact same conditions as the object 104 under examination. However, in certain implementations, it may be impractical to place the reflectance standard 112 in the same optical plane as the object 104 under examination. Moreover, commercial reflectance standards 112 are generally designed as flat reflective targets. Such flat reflective targets can be a poor approximation for the object 104 under examination when the object 104 under examination is not a flat surface. Therefore, if the object 104 under examination is not a flat surface or the reflectance standard 112 is not placed at the same optical plane as the object 104 under examination, the accuracy and performance of the above correction formula is significantly reduced, leading to loss of spectral reflectance measurement accuracy when imaging the object 104 under examination. As a non-limiting example, in some instances, the object 104 under examination may be a retina of an eye. In some instances, the object 104 under examination may be another part of an eye.

Indeed, the above-noted difficulties in spectral calibration are particularly evident when the object 104 under examination is a retina of an eye. The eye is a natural optical system with its own lens and refractive power. The retina is located at the back of the eye behind optical elements such as, but not limited to, the cornea, crystalline lens, aqueous and vitreous humors, sclera, and dynamic tear film. Therefore, the retina is located in an optical plane having a varying position or distance depending on the particular eye being imaged (e.g. myopic, emmetropic, and hyperopic eyes). However, it may be difficult or undesirable to implant the reflectance standard 112 in the eye at the optical plane of the retina in order to generate a reference measurement for subsequent spectral imaging of the eye.

Moreover, even if the reflectance standard 112 were implanted in the eye in the same optical plane as the retina, the retina is a curved surface—the average adult retina having a radius of curvature of about 11 mm. The retina curvature is functional to the imaging properties of all optical lenses. When an object under visual study lies on a plane orthogonal to the optical axis of the eye, real lenses have a curved in-focus image surface, and every image point that lies outside of the optical axis is affected by the, so called, field curvature. The retina is a curved surface to overcome this effect, allowing points outside the optical axis of the eye to be in focus. Referring to FIG. 1C, which shows an ophthalmic imaging system for reforming a field curvature corrected image on an imaging sensor, ophthalmic imaging systems capture an image of a retina on a planar sensor. Therefore, an ophthalmic imaging sensor may be tuned and designed to correct for the retinal curvature. For instance, FIG. 1C shows two emitting sources at different positions on the imaged field that are in focus on the imaging sensor regardless of their position and distance from the optical axis. The curvature of the retina as well as the optical elements of the eye such as the cornea and crystalline lens, may be considered to have a sharp, aberration-free, in-focus image on the sensor. Therefore, precise and tailored optical components of ophthalmic imaging systems are designed to project the curved surface of the retina on a planar sensor. During calibration, if a flat reflectance standard is used instead of a curved one, its image will be affected by field curvature and points outside of the optical axis of the imaging system will be out of focus. In spectral imaging systems, out-of-focus points lead to spatial and spectral cross-correlation among different objects on the scene that is imaged. Indeed, out-of-focus points on the image plane spread over a larger area than focused points and, therefore, overlap the focused points, contaminating the spectral information of neighboring pixels. Accordingly, flat reflectance standard targets are limited in their ability to approximate the reflectance of the curved retina.

Based on the above discussion, it can be seen that the optical elements of the eye and the curvature of the retina combine to modify the light incident upon and reflected from the retina with absorptions, scattering, optical aberrations, and refractions that are not accounted for by a typical flat reflectance standard 112 positioned outside of the eye. Therefore, there is a need for spectral calibration systems that include phantom, or artificial, eyes. Such phantom eyes may eliminate the need to implant a reflectance standard in a biological eye under examination. Such phantom eyes may include curved reflectance standards for more accurately approximating or mimicking the retina of a biological eye. To avoid or reduce the above-described out-of-focus cross-correlation among pixels during calibration of spectral systems, the phantom eye curvature and optics can be as close as possible to that of a real biological eye under study.

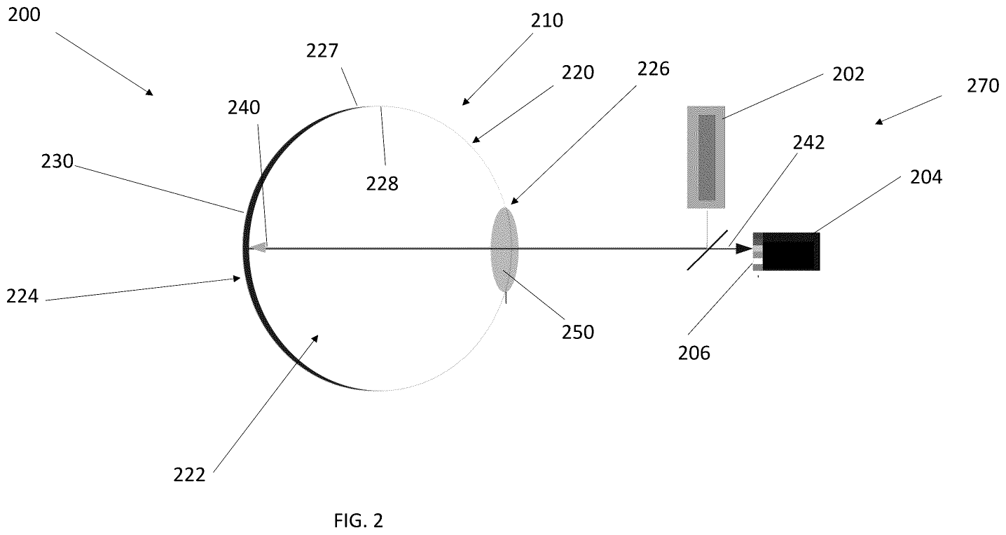

Referring now to FIG. 2, a spectral calibration system 200 is depicted. In some embodiments, the spectral calibration system 200 can include a lighting assembly 270, which can include a light source 202 and a sensor 204. In some embodiments, the light source 202 can be a broadband light source, which emits a wide spectrum of light, for example, in the UV, visible, near infrared, or infrared wavelength ranges, or it may be a narrowband light source which emits a narrow spectrum or single wavelength of light. In some embodiments, the light source 202 can emit a single continuous spectrum of light or it can emit more than one discontinuous spectra. In some embodiments, the light source 202 can emit light with a constant intensity over the wavelength range or the wavelengths and intensity can be adjustable. In some embodiments, the light source 202 can be comprised of a single light source or a combination of multiple light sources of the same or different types described above. In some embodiments, the light source 202 can be a xenon lamp, a mercury lamp, an LED, a laser, superluminescent diodes, supercontinuum light sources, or any other light source. In some embodiments, the sensor 204 can be any suitable sensor for spectral imaging, including but not limited to, a monochrome camera, a camera with a mosaic filter array, a point spectrophotometer, or single-pixel detector for Scanning Laser Ophthalmoscopy (SLO)-type systems, such as a diode/PMT. In some embodiments, the sensor 204 includes a spectral filter 206 to filter light detected by the sensor 204. In some embodiments, the sensor 204 can be filterless, and the filtering of light can be done in the light source 202 or elsewhere along the optical path between the light source 202 and the sensor 204. The filter 206 can filter light entering the sensor 204, such that only selected wavelengths of light are received by the sensor 204.

The spectral calibration system 200 further includes a phantom eye 210. The phantom eye 210 can include a shell 220 and a reflectance standard 230. In some embodiments, the shell 220 can include one or more curved surfaces defining an internal volume 222 of the phantom eye 210. In some embodiments, the shell 220 can include at least a first curved portion 224 and a second curved portion 226. In some embodiments, the first curved portion 224 and the second curved portion 226 are in line with the optical path of light 240 emitted by the light source 202 to the reflectance standard 230. In some embodiments, the first curved portion 224 and the second curved portion 226 are in line with the optical path of light 242 reflected by the reflectance standard 230 to the sensor 204. In some embodiments, the first curved portion 224 and the second curved portion 226 may be diametrically opposite each other. In some embodiments, the first curved portion 224 is a posterior portion of the shell 220 of the phantom eye 210. In some embodiments, the second curved portion 226 is an anterior portion of the shell 220 of the phantom eye 210. In some embodiments, the shell 220 can include an outer surface 227 and an inner surface 228. In some embodiments, one or more components of the phantom eye 210 mimicking the structure or function of components of a biological eye can be secured to the shell 220 at or along the outer surface 227 or the inner surface 228. In some embodiments, one or more components of the phantom eye 210 mimicking the structure or function of components of a biological eye can be secured within the internal volume 222 of the phantom eye 210 at or along the outer surface 227 or the inner surface 228.

The reflectance standard 230 can be curved to mimic the structure or function of a retina. The reflectance standard 230 can mimic an optical property of the retina. “Optical property,” as used herein can refer to any property that defines how a material interacts with light. In some embodiments, optical properties can include one or more of, but are not limited to, curvature, refractive or optical power, and transmittance. In some embodiments, the reflectance standard 230 can have a radius of curvature of about 11 mm. In some embodiments, the reflectance standard 230 can have a radius of curvature in a range of 7 mm to 15 mm. In some embodiments, the reflectance standard 230 can have a radius of curvature in a range of 10 mm to 12 mm. In some embodiments, the reflectance standard 230 can, itself, define a posterior surface of the phantom eye 210. In such embodiments, the shell 220 can be segmented such that the shell 220 and the reflectance standard 230, in combination, at least partially define the internal volume 222 of the phantom eye 210. In some embodiments, the reflectance standard 230 can be secured within the internal volume 222 of the phantom eye 210 at or along the inner surface 228 of the shell 220. In some embodiments, the reflectance standard 230 can be coated on the inner surface 228 of the shell 220. In some embodiments, the reflectance standard 230 can be secured to the inner surface 228 of the first curved portion 224 of the shell 220. In some embodiments, the reflectance standard 230 can be coated on the inner surface 228 of the first curved portion 224 of the shell 220. In some embodiments, the reflectance standard 230 can be white. In some embodiments, the reflectance standard 230 can be made from or coated with reflectance standard materials or surface coatings with known reflectance, such as but not limited to, Spectralon™, Permaflext, BaSO4, or PTFE. In some embodiments, the reflectance standard 230 can be a different color than white. In some embodiments, the reflectance standard 230 can have more than one color on the same surface. In some embodiments, the reflectance standard 230 can be fluorescent or have some biological elements or artificial elements on its surface. In some embodiments, the reflectance standard 230 can be made up of several layers of material of varying or equal thickness, which can scale from nanometer to centimeter. In some embodiments, the reflectance standard 230 can generally include a light-receiving surface configured to be illuminated by light. In some embodiments, the light-receiving surface can be configured to reflect light back to a sensor. In some embodiments, the light-receiving surface can be a concave surface of the reflectance standard 230.

In some embodiments, the phantom eye 210 can include ocular media 250. In some embodiments, the ocular media 250 can include one or more custom ocular components that enable fine-grained control over the optical properties of the phantom eye 210. The optical properties can include, among others, spectral transmittance and optical power of the phantom eye 210. In some embodiments, the ocular media 250 can include one or more components mimicking the structure and function of components of a biological eye. In some embodiments, the ocular media 250 can include one or more components mimicking the optical properties, such as optical power, of a biological eye. In some embodiments, the ocular media 250 can be positioned between the reflectance standard 230 and the light source 202. In some embodiments, the ocular media 250 can be positioned between the reflectance standard 230 and the sensor 204. In some embodiments, the ocular media 250 can be positioned in or on the phantom eye 210 anterior of the reflectance standard 230. In some embodiments, one or more components of the ocular media 250 can define an anterior surface of the phantom eye 210. In such embodiments, the shell 220 can be segmented such that the shell 220 and the ocular media 250, in combination, at least partially define the internal volume 222 of the phantom eye 210. In some embodiments, one or more components of the ocular media 250 can be secured within the internal volume 222 of the phantom eye 210 at or along the inner surface 228 of the shell 220. In some embodiments, one or more components of the ocular media 250 can be secured to the inner surface 228 of the second curved portion 226 of the shell 220. In some embodiments, one or more components of the ocular media 250 can be secured at or along the outer surface 227 of the shell 220. In some embodiments, one or more components of the ocular media 250 can be secured to the outer surface 227 of the second curved portion 226 of the shell 220. In some embodiments, the ocular media 250 can include the shell 220. That is, the shell 220 can be made of such a material and shaped to be a custom ocular component that enables fine-grained control over the spectral transmittance of the phantom eye 210.

The ocular media 250 can alter the spectral radiant emittance from the light source 202 to the reflectance standard 230. In some embodiments, the ocular media 250 can be designed to have custom, known spectral characteristics. These custom characteristics can be derived from biometric optical coherence tomography (anterior segment OCT) acquisitions, visible light hyperspectral optical coherence tomography (vis-OCT), psychophysical measurement, Purkinje imaging, technical datasheets of intraocular replacement lens manufacturers, or other sources. The ocular media 250 can include components to mimic a biological eye's crystalline lens, cornea, or other optical elements of the biological eye. The phantom eye 210 can include any number or type of ocular media 250 components, up to the phantom eye 210 being an entire optical replica of a biological eye. The number and type of components of the ocular media 250 implemented in the phantom eye 210 and the design of each of the implemented components may be particularly selected such that the transmittance, refractive power, or other optical property of the ocular media 250 is particularly controlled. Particularly, the design of each of the implemented components can be particularly selected to mimic the optical properties, such as optical or refractive power, of a biological eye.

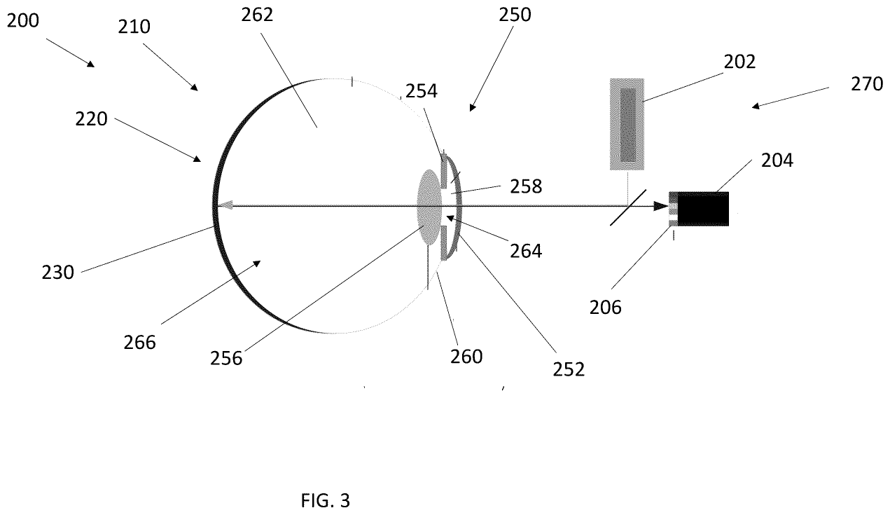

Reference is now made to FIG. 3, which depicts sub-components of the ocular media 250 of the phantom eye 210 of the spectral calibration system 200, in conjunction with FIG. 2. In some embodiments, the ocular media 250 can include an artificial sclera 260. The artificial sclera 260 can be made of Polycaprolactone (PCL), any glass, Polymethyl-methacrylate (PMMA), or other suitable polymers, for example. In some embodiments, the artificial sclera 260 can define the shell 220 discussed with respect to FIG. 2. That is, in some embodiments, the artificial sclera 260 can form the shell 220, including any or all of the first curved portion 224, the second curved portion 226, the outer surface 227, and the inner surface 228. The artificial sclera 260 can be particularly included in the ocular media 250 for hyper/multispectral calibration systems with transscleral illumination. In some embodiments, at least a portion of the artificial sclera 260 is positioned between the light source 202 of the lighting assembly 270 and the reflectance standard 230 along the optical path of light 240 emitted from the light source 202 to the reflectance standard 230. In some embodiments, at least a portion of the artificial sclera 260 is positioned between the reflectance standard 230 and the sensor 204 of the lighting assembly 270 along the optical path of light 242 reflected to the sensor 204 from the reflectance standard 230. In some embodiments, at least a portion of the artificial sclera 260 is positioned anterior the reflectance standard 230. In some embodiments, the artificial sclera 260 can mimic optical properties of a biological sclera. In some embodiments, the artificial sclera 260 can mimic physical properties of a biological sclera.

In some embodiments, the ocular media 250 can include an artificial cornea 252. In some embodiments, the artificial cornea 252 can be made of polydimethylsiloxane (PDMS), any glass, Polymethyl-methacrylate (PMMA), or other suitable polymers, for example. In some embodiments, the artificial cornea 252 can be connected or adhered to an outer surface of the artificial sclera 260 or shell 220. For instance, in some embodiments, the artificial cornea 252 can be connected or adhered to the outer surface 227. In some embodiments, the artificial sclera 260 or shell 220 can be segmented such that an opening is formed in an anterior portion of the artificial sclera 260 or shell 220, and the artificial cornea 252 can bridge the opening formed in the anterior portion of the artificial sclera 260 or shell 220. In some embodiments, the artificial cornea 252 is positioned between the light source 202 of the lighting assembly 270 and the reflectance standard 230 along the optical path of light 240 emitted from the light source 202 to the reflectance standard 230. In some embodiments, the artificial cornea 252 is positioned between the reflectance standard 230 and the sensor 204 of the lighting assembly 270 along the optical path of light 242 reflected to the sensor 204 from the reflectance standard 230. In some embodiments, the artificial cornea 252 is positioned anterior the reflectance standard 230. Including the artificial cornea 252 in the ocular media 250 of the phantom eye 210 can be useful for adding pathological formations and shapes or spectrum-altering effect, particularly in simulating or mimicking biological eyes with disorders such as keratoconus. In some embodiments, the artificial cornea 252 can be formed by a series of layers with single or different refractive indexes to mimic a biological eye. In some embodiments, the artificial cornea 252 can mimic optical properties of a biological cornea. In some embodiments, the artificial cornea 252 can mimic physical properties of a biological cornea.

In some embodiments, the ocular media 250 can include an artificial iris 254. In some embodiments, the artificial iris 254 can be made of, for example, commercial lens apertures. In some embodiments, the artificial iris 254 can be made of, for example, a fluidic system based on electrowetted-actuated mixtures of opaque and transparent fluids, totally black and absorptive materials, or totally reflective materials. In some embodiments, the artificial iris 254 can be a diaphragm that allows light to transmit only on a portion of its internal or external area. In some embodiments, the artificial iris 254 can be connected or adhered to an inner surface of the artificial sclera 260 or shell 220 or an inner surface of the artificial cornea 252. In some embodiments, the artificial iris 254 is positioned between the artificial cornea 252 and the reflectance standard 230 along the optical path of light 240 emitted from the light source 202 to the reflectance standard 230 or the optical path of light 242 reflected to the sensor 204 from the reflectance standard 230. In some embodiments, the artificial iris 254 is positioned anterior the reflectance standard 230. In some embodiments the artificial iris 254 is positioned posterior the artificial cornea 252. In some embodiments, the artificial iris 254 is positioned between the light source 202 of the lighting assembly 270 and the reflectance standard 230 along the optical path of light 240 emitted from the light source 202 to the reflectance standard 230. In some embodiments, the artificial iris 254 is positioned between the reflectance standard 230 and the sensor 204 of the lighting assembly 270 along the optical path of light 242 reflected to the sensor 204 from the reflectance standard 230. The artificial iris 254 can be included in the ocular media 250 to simulate or mimic the effect of differently sized pupils (e.g., senile miosis in elderly population) and how pupil dilation (for instance, with tropicamide) affects the spectral image quality. In some embodiments, the ocular media 250 can include a plurality of artificial irises 254. In some embodiments, the artificial iris 254 can be a tunable diaphragm that can allow more or less light to pass through to the reflectance standard 230, or can allow more or less light to pass through to the sensor 204. In some embodiments, the artificial iris 254 can mimic optical properties of a biological iris. In some embodiments, the artificial iris 254 can mimic physical properties of a biological iris.

In some embodiments, the ocular media 250 can include an artificial crystalline lens 256. In some embodiments, the artificial crystalline lens 256 can be made of, for example, commercially available lenses or custom-manufactured lenses. In some embodiments, the artificial crystalline lens 256 can be made of polydimethylsiloxane (PDMS), any glass, Polymethyl-methacrylate (PMMA), or other suitable polymers, for example. In some embodiments, the custom-manufactured lenses can have spectral absorbers and scatterers mimicking the lens yellowing (accumulation and aggregation of crystallins) of older subjects. In some embodiments, the artificial crystalline lens 256 can be connected or adhered to an inner surface of the artificial sclera 260 or shell 220 or an inner surface of the artificial iris 254. In some embodiments, the artificial crystalline lens 256 can be positioned between the artificial cornea 252 or artificial iris 254 and the reflectance standard 230 along the optical path of light 240 emitted from the light source 202 to the reflectance standard 230 or the optical path of light 242 reflected to the sensor 204 from the reflectance standard 230. In some embodiments, the artificial crystalline lens 256 is positioned anterior the reflectance standard 230. In some embodiments, the artificial crystalline lens 256 is positioned posterior the artificial cornea 252 or artificial iris 254. In some embodiments, the artificial crystalline lens 256 can be positioned anterior the artificial iris 254. In some embodiments, the artificial crystalline lens 256 is positioned between the light source 202 of the lighting assembly 270 and the reflectance standard 230 along the optical path of light 240 emitted from the light source 202 to the reflectance standard 230. In some embodiments, the artificial crystalline lens 256 is positioned between reflectance standard 230 and the sensor 204 of the lighting assembly along the optical path of light 242 reflected to the sensor 204 from the reflectance standard 230. In some embodiments, the artificial crystalline lens 256 can be an intraocular lens. In some embodiments, the artificial crystalline lens 256 can have different optical powers than emmetropic eyes. In some embodiments, the artificial crystalline lens 256 can be a plano-concave, pano-convex, meniscus, a double convex, or double concave lens. In some embodiments, the artificial crystalline lens 256 can mimic optical properties of a biological crystalline lens. In some embodiments, the artificial crystalline lens 256 can mimic physical properties of a biological crystalline lens.

In some embodiments, the ocular media 250 can include artificial aqueous humor 258. In some embodiments, the artificial aqueous humor 258 can be made of, for example, polymetric fluid, water, or any other liquid or polymer that match the refractive index and spectral properties of human aqueous humor. In some embodiments, the artificial aqueous humor 258 can be located within a cavity 264 defined by at least portions of the artificial cornea 252, the artificial iris 254, or the artificial crystalline lens 256. In some embodiments, the artificial aqueous humor 258 is located between the artificial cornea 252 and the artificial crystalline lens 256 or reflectance standard 230 along the optical path of light 240 emitted from the light source 202 to the reflectance standard 230 or the optical path of light 242 reflected to the sensor 204 from the reflectance standard 230. In some embodiments, the artificial aqueous humor 258 is positioned anterior the reflectance standard 230 and the artificial crystalline lens 256. In some embodiments, the artificial aqueous humor 258 is positioned posterior the artificial cornea 252. In some embodiments, the artificial aqueous humor 258 is located between the light source 202 of the lighting assembly 270 and the reflectance standard 230 along the optical path of light 240 emitted from the light source 202 to the reflectance standard 230. In some embodiments, the artificial aqueous humor 258 is located between the reflectance standard 230 and the sensor 204 of the lighting assembly 270 along the optical path of light 242 reflected to the sensor 204 from the reflectance standard 230. In some embodiments, the artificial aqueous humor 258 can contain particles and can mimic pathological conditions. In some embodiments, the artificial aqueous humor 258 internal pressure can be controlled to mimic pathological states such as glaucoma. In some embodiments, the artificial aqueous humor 258 can mimic optical properties of a biological aqueous humor. In some embodiments, the artificial aqueous humor 258 can mimic physical properties of a biological aqueous humor.

In some embodiments, the ocular media 250 can include artificial vitreous humor 262. In some embodiments, the artificial vitreous humor 262 can be made of, for example, polymeric fluid, water, or any other material that mimics the spectral properties and optical properties of the human vitreous. In some embodiments, the artificial vitreous humor 262 can be located within a cavity 266 defined by at least portions of the reflectance standard 230, the artificial crystalline lens 256, and the artificial sclera 260 or shell 220. In some embodiments, the artificial vitreous humor 262 is positioned between the reflectance standard 230 and the artificial crystalline lens 256 along the optical path of light 240 emitted from the light source 202 to the reflectance standard 230 or the optical path of light 242 reflected to the sensor 204 from the reflectance standard 230. In some embodiments, the artificial vitreous humor 262 is positioned anterior the reflectance standard 230. In some embodiments, the artificial vitreous humor 262 is positioned posterior the artificial crystalline lens 256. In some embodiments, the artificial vitreous humor 262 is positioned between the light source 202 of the lighting assembly 270 and the reflectance standard 230 along the optical path of light 240 emitted from the light source 202 to the reflectance standard 230. In some embodiments, the artificial vitreous humor 262 is positioned between the reflectance standard 230 and the sensor 204 of the lighting assembly 270 along the optical path of light 242 reflected to the sensor 204 from the reflectance standard 230. In some embodiments, the artificial vitreous humor 262 can mimic optical properties of a biological vitreous humor. In some embodiments, the artificial vitreous humor 262 can mimic physical properties of a biological vitreous humor.

As noted above, the number and type of components of the ocular media 250 implemented in the phantom eye 210 and the design of each of the implemented components can be particularly selected such that the transmittance and refractive power of the ocular media 250 is particularly controlled. The below equations represent the relationships between the spectral characteristics of the light source 202 (represented by I(λ)), which may be known, the spectral characteristics of the ocular media 250 (represented by M(λ), or more specifically 2M(λ) for a double-pass reflectance measurement), which may be particularly selected, the spectral characteristics of the reflectance standard 230 (represented by R(λ)), which may be known, the spectral characteristics of the spectral filter 206 (represented by F(λ)), which may be known, and the spectral characteristics of light measured by the sensor 204 (represented by S(λ)). It should be appreciated that the ocular media 250 parameters of spectral characteristics M(λ) may be further split into subfunctions. Each of the subfunctions may represent the spectral characteristics of a single component of the ocular media 250, for instance.

$$S ( λ ) = I ( λ ) \cdot 2 M ( λ ) \cdot R ( λ ) \cdot F ( λ ) \\ R ( λ ) = \frac{S ( λ )}{I ( λ ) \cdot 2 M ( λ ) \cdot F ( λ )} \\ F ( λ ) = \frac{S ( λ )}{I ( λ ) \cdot 2 M ( λ ) \cdot R ( λ )}$$

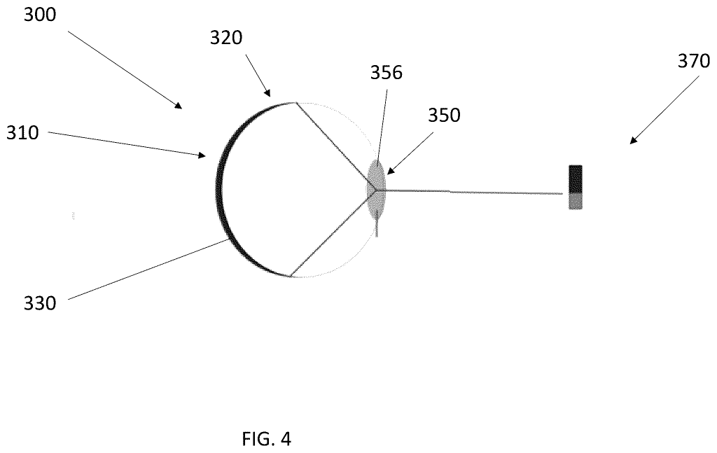

It should be appreciated that the ocular media 250 of the phantom eye 210 can include more or fewer components than those discussed with respect to FIG. 3. For instance, the ocular media 250 can include tear film of other optical components to simulate or mimic the optics of a biological eye. Moreover, the phantom eye can include various components of the ocular media 250 in any desired combination. For instance, referring to FIG. 4, a calibration system 300 is depicted. The calibration system 300 includes a phantom eye 310, shown in cross section. The phantom eye 310 can resemble the phantom eye 210 discussed with respect to FIGS. 2 and 3 in all aspects except as noted herein. For instance, the phantom eye 310 can include a shell 320 which, in some embodiments, can be made of artificial sclera, and a reflectance standard 330. The phantom eye 310 further includes ocular media 350, which can include an artificial crystalline lens 356 without any or all of the artificial cornea 252, artificial iris 254, artificial vitreous humor 262, artificial sclera 260, or artificial aqueous humor 258 discussed with respect to FIG. 3. In some embodiments, the artificial crystalline lens 356 can be connected or adhered to an outer surface of the shell 320. In some embodiments, the artificial crystalline lens 356 can be connected or adhered to an inner surface of the shell 320. In some embodiments, the shell 320 can be segmented such that an opening is formed in an anterior portion of the shell 320, and the artificial crystalline lens 356 can bridge the opening formed in the anterior portion of the shell 320. The calibration system 300 can further include a lighting assembly 370, which is depicted in a simplified form but can resemble the lighting assembly 270 (FIGS. 2 and 3) and include a light source and a sensor. As discussed with respect to FIG. 3, in some embodiments, the artificial crystalline lens 356 is positioned between the light source of the lighting assembly 370 and the reflectance standard 330 along the optical path of light emitted from the light source of the lighting assembly 370 to the reflectance standard 330. In some embodiments, the artificial crystalline lens 356 is positioned between the reflectance standard 330 and the sensor of lighting assembly 370 along the optical path of light reflected to the sensor of the lighting assembly 370 from the reflectance standard 330. In some embodiments, the artificial crystalline lens 356 is positioned anterior the reflectance standard 330. The phantom eye 310 can include the reflectance standard 330 coated on an interior surface of the shell 320, as mentioned with respect to FIGS. 2 and 3.

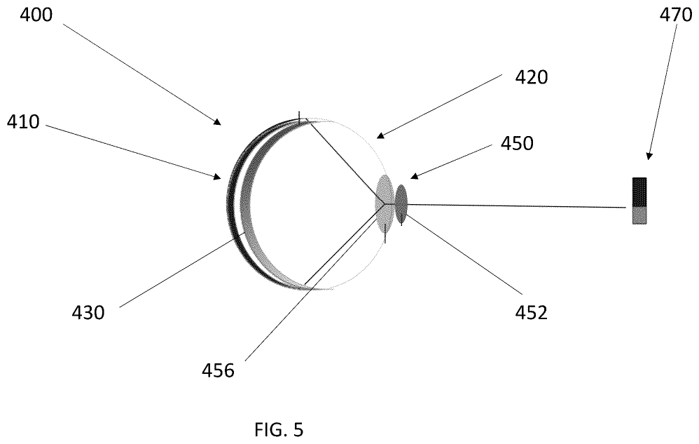

Referring to FIG. 5, a calibration system 400 is depicted. The calibration system 400 includes a phantom eye 410, shown in cross section. The phantom eye 410 can resemble the phantom eye 210 discussed with respect to FIGS. 2 and 3 in all aspects except as noted herein. For instance, the phantom eye 410 can include a shell 420 which, in some embodiments, may be made of artificial sclera, and a reflectance standard 430. The phantom eye 410 further includes ocular media 450, which can include an artificial crystalline lens 456 and an artificial cornea 452 without any or all of the artificial iris 254, artificial vitreous humor 262, artificial sclera 260, or artificial aqueous humor 258 discussed with respect to FIG. 3. In some embodiments, the artificial crystalline lens 456 can be connected or adhered to an outer surface of the shell 420. In some embodiments, the artificial crystalline lens 456 can be connected or adhered to an inner surface of the shell 420. In some embodiments, the shell 420 can be segmented such that an opening is formed in an anterior portion of the shell 420, and the artificial crystalline lens 456 can bridge the opening formed in the anterior portion of the shell 420. In some embodiments, the artificial cornea 452 can be connected or adhered to an outer surface of the shell 420. In some embodiments, the artificial cornea 452 can be connected or adhered to an inner surface of the shell 420. In some embodiments, the shell 420 can be segmented such that an opening is formed in an anterior portion of the shell 420, and the artificial cornea 452 can bridge the opening formed in the anterior portion of the shell 420. In some embodiments, the artificial cornea 452 can be connected or adhered to an anterior surface of the artificial crystalline lens 456.

The calibration system 400 can further include a lighting assembly 470, which is depicted in a simplified form but can resemble the lighting assembly 270 (FIGS. 2 and 3) and include a light source and a sensor. In some embodiments, the artificial crystalline lens 456 and artificial cornea 452 are located between the light source of the lighting assembly 470 and the reflectance standard 430 along the optical path of light emitted from the light source of lighting assembly 470 to the reflectance standard 430. In some embodiments, the artificial crystalline lens 456 and artificial cornea 452 are located between the reflectance standard 430 and the sensor of the lighting assembly 470 along the optical path of light reflected to the sensor from the reflectance standard 430. In some embodiments, the artificial crystalline lens 456 and artificial cornea 452 are positioned anterior the reflectance standard 430. In some embodiments, the artificial crystalline lens 456 is positioned anterior the reflectance standard 430 and posterior the artificial cornea 452.

Still referring to FIG. 5, the phantom eye 410 can include an interchangeable reflectance standard 430. For instance, in some embodiments, the shell 420 can include one or more receptors, such as, at least partial, grooves, recesses, or teeth along an inner surface of the shell 420. A user can then insert a select reflectance standard 430 into the receptors by means of a friction fit or snap fit, for instance, between the reflectance standard 430 and the receptors. Therefore, a user can interchange reflectance standards 430 in the phantom eye 410, as desired, selecting a reflectance standard 430 with a particular curvature, for instance, that mimics or mirrors the curvature of a retina of a biological eye of interest. It should be appreciated that any of the above-described embodiments of phantom eyes can similarly include an interchangeable reflectance standard 430. Moreover, it should be appreciated that the phantom eye 410 can include a reflectance standard 430 that is coated on or fixedly secured to the shell 420.

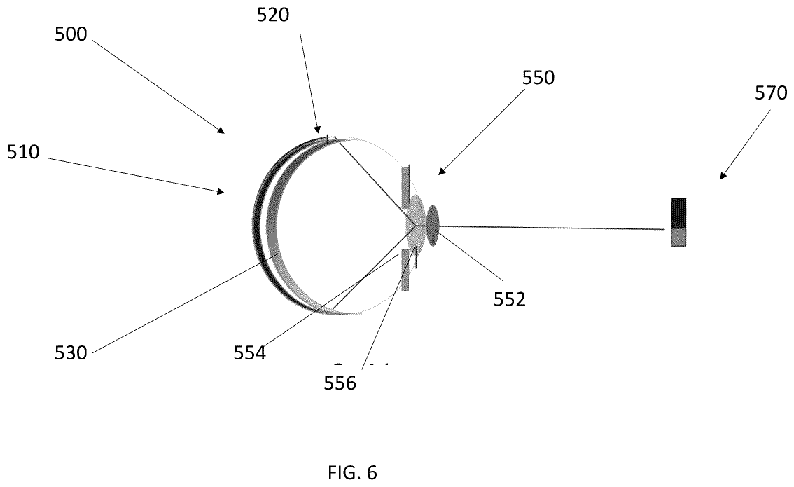

Referring to FIG. 6, a calibration system 500 is depicted. The calibration system 500 includes a phantom eye 510, shown in cross section. The phantom eye 510 can resemble the phantom eye 210 discussed with respect to FIGS. 2 and 3 in all aspects except as noted herein. For instance, the phantom eye 510 can include a shell 520 which, in some embodiments, can be made of artificial sclera, and a reflectance standard 530. The phantom eye 510 further includes ocular media 550, which can include an artificial crystalline lens 556, an artificial cornea 552, and an artificial iris 554 without any or all of the artificial vitreous humor 262, artificial sclera 260, or artificial aqueous humor 258 discussed with respect to FIG. 3.

In some embodiments, the artificial crystalline lens 556 can be connected or adhered to an outer surface of the shell 520. In some embodiments, the artificial crystalline lens 556 can be connected or adhered to an inner surface of the shell 520. In some embodiments, the shell 520 can be segmented such that an opening is formed in an anterior portion of the shell 520, and the artificial crystalline lens 556 can bridge the opening formed in the anterior portion of the shell 520. In some embodiments, the artificial cornea 552 can be connected or adhered to an outer surface of the shell 520. In some embodiments, the artificial cornea 552 can be connected or adhered to an inner surface of the shell 520. In some embodiments, the shell 520 can be segmented such that an opening is formed in an anterior portion of the shell 520, and the artificial cornea 552 can bridge the opening formed in the anterior portion of the shell 520. In some embodiments, the artificial cornea 552 can be connected or adhered to an anterior surface of the artificial crystalline lens 556. In some embodiments, the artificial iris 554 can be connected or adhered to an inner surface of the shell 520. In some embodiments, the artificial iris 554 can be connected or adhered to a posterior surface of the crystalline lens 556.

The calibration system 500 can further include a lighting assembly 570, which is depicted in a simplified form but can resemble the lighting assembly 270 (FIGS. 2 and 3) and include a light source and sensor. In some embodiments, the artificial crystalline lens 556, artificial cornea 552, and artificial iris 554 are located between the light source of the lighting assembly 570 and the reflectance standard 530 along the optical path of light emitted from the light source of the lighting assembly 570 to the reflectance standard 530. In some embodiments, the artificial crystalline lens 556, artificial cornea 552, and artificial iris 554 are positioned between the reflectance standard 530 and the sensor of the lighting assembly 570 along the optical path of light reflected to the sensor from the reflectance standard 530. In some embodiments, the artificial crystalline lens 556, artificial cornea 552, and artificial iris 554 are positioned anterior the reflectance standard. In some embodiments, the artificial iris 554 is positioned anterior the reflectance standard 530, and the artificial crystalline lens is positioned anterior the artificial iris 554 and posterior the artificial cornea 552. While the phantom eye 510 is depicted having an interchangeable reflectance standard 530, it should be appreciated that the phantom eye 510 can instead include a reflectance standard 530 that is coated on or fixedly secured to the shell 520.

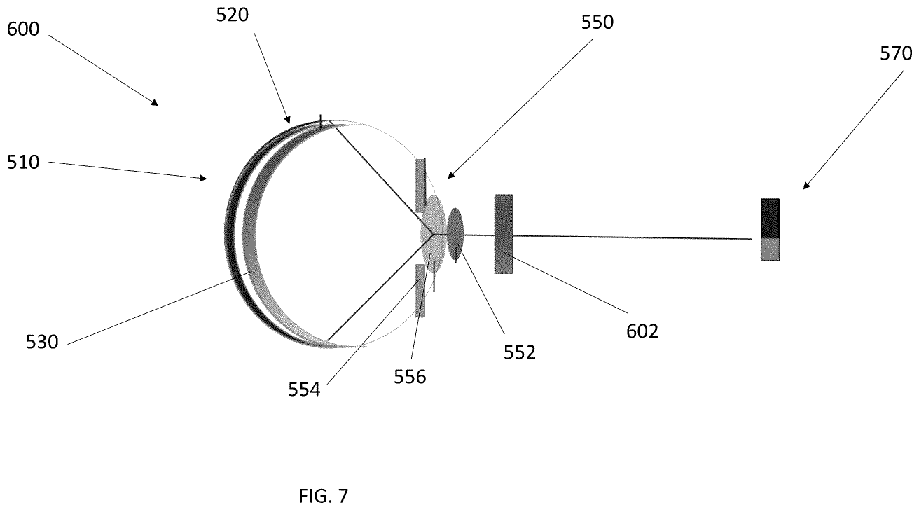

Referring now to FIG. 7, a calibration system 600 is depicted. In some embodiments, the calibration system 600 can include the phantom eye 510, shown in cross section and discussed with respect to FIG. 6. In some embodiments, the calibration system 600 can further include the lighting assembly 570 discussed with respect to FIG. 6. In some embodiments, the calibration system 600 can further include a spectral filter 602. The spectral filter 602 can attenuate particular wavelengths of light. For instance, if a light source of the lighting assembly 570 does not emit many photons at a certain wavelength range, a spectral reflectance image of the phantom eye 510 may appear noisy. Therefore, the spectral filter 602 can be included in the calibration system 600 to attenuate the wavelengths that would otherwise dominate the spectral power distribution (SPD) of the light source of the lighting assembly 570. In some embodiments, the spectral filter 602 can be a flattening filter. In some embodiments, the spectral filter 602 can compensate for system transmittance. In some embodiments, the spectral filter 602 can account for standard observer ocular media absorption and fundus reflectance.

In some embodiments, the spectral filter 602 can be connected or adhered to an outer surface of the shell 520. In some embodiments, the spectral filter 602 can be connected or adhered to an inner surface of the shell 520. In some embodiments, the spectral filter 602 can be connected or adhered to the most anterior component of the ocular media 550. For instance, in some embodiments, the spectral filter 602 can be connected or adhered to an anterior surface of the artificial cornea 552. In some embodiments, such as those featuring the phantom eye 310 depicted in FIG. 4, the spectral filter 602 can be connected or adhered to an anterior surface of the artificial crystalline lens 356 (FIG. 4). In some embodiments, the spectral filter 602 may not be physically coupled to the phantom eye 510. In some embodiments, the spectral filter 602 is positioned between the light source of the lighting assembly 570 and the reflectance standard 530 along the optical path of light emitted from the light source of the lighting assembly 570 to the reflectance standard 530. In some embodiments, the spectral filter 602 is positioned between the reflectance standard 530 and the sensor of the lighting assembly 570 along the optical path of light reflected to the sensor of the lighting assembly 570 from the reflectance standard 530. In some embodiments, the spectral filter 602 can be positioned anterior the reflectance standard 530. In some embodiments, the spectral filter 602 can be positioned between the lighting assembly 570 and the ocular media 550 along the optical path of light emitted from the light source of the lighting assembly 570 to the reflectance standard 530. In some embodiments, the spectral filter 602 can be positioned between the lighting assembly 570 and the ocular media 550 along the optical path of light reflected to the sensor of the lighting assembly 570 from the reflectance standard 530. In some embodiments, the spectral filter 602 can be positioned anterior the ocular media 550. It should be appreciated that the spectral filter 602 can be incorporated in any of the previously or subsequently described calibration systems.

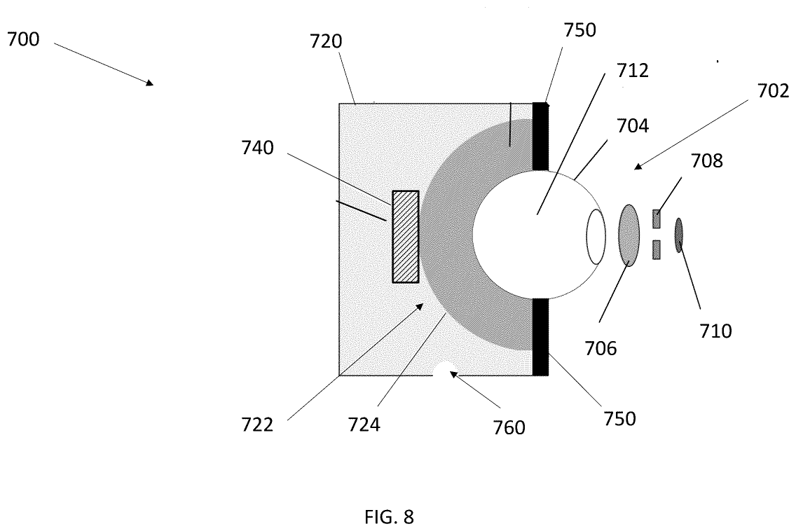

Referring now to FIG. 8, a cross section of a phantom eye 700 is depicted. The phantom eye 700 can resemble the phantom eye 210 discussed with respect to FIGS. 2 and 3 in all aspects except as noted herein. For instance, the phantom eye 700 can include a shell, which, in some embodiments, can be made of artificial sclera, and a reflectance standard 730. The phantom eye 700 can further include ocular media 702, which can include an artificial sclera 704, an artificial crystalline lens 706, an artificial iris 708, an artificial cornea 710, or artificial vitreous humor 712. It should be appreciated that the ocular media 702 can include any combination of ocular components discussed with respect to FIGS. 2-7. The ocular media 702 is shown in an exploded view for ease of illustration. However, it should be appreciated that one or more components of the ocular media 702 can be assembled on the phantom eye 700 similar to the ocular media discussed with respect to FIGS. 2-7.

In some embodiments, the phantom eye 700 can include an enclosure 720. The enclosure 720 can house one or more sub-components of the phantom eye 700, including the ocular media 702 and the reflectance standard 730. In some embodiments, the enclosure 720 can include a curved cutout 722 within which the reflectance standard 730 and/or one or more components of the ocular media 702 are positioned. In some embodiments, the reflectance standard 730 is positioned against a surface 724 of the cutout 722. In some embodiments, the reflectance standard 730 is coupled or adhered to the surface 724 of the cutout 722. In some embodiments, one or more components of the ocular media 702, such as the sclera 704 may be positioned against an anterior surface of the reflectance standard 730 and within the curved cutout 722. In some embodiments, one or more components of the ocular media 702 may be coupled to the anterior surface of the reflectance standard 730.

In some embodiments, an actuator 740 is positioned within the enclosure 720. In some embodiments, the actuator 740 can be a stage, such as a six-degrees-of-freedom stage, a mounting bob, or other mechanical actuator. In some embodiments, the actuator 740 can be coupled to the reflectance standard 730. In some embodiments, the actuator 740 can be coupled to a posterior surface of the reflectance standard 730. In some embodiments, at least a portion of the actuator 740 can be extend along or through an opening in the surface 724 of the cutout 722. In some embodiments, the actuator 740 can adjust the positioning of the reflectance standard 730 and one or more components of the ocular media 702 coupled to the reflectance standard 730 relative to the cutout 722 and the enclosure 720. In some embodiments, the actuator 740 can adjust the position of the reflectance standard 730 and one or more components of the ocular media 702 for aligning a particular entrance of the phantom eye 700 with a sensor and/or light source of a lighting assembly.

In some embodiments, the mechanical enclosure 720 includes a mounting member 760 in the enclosure 720. In some embodiments, the mounting member 760 can be formed in an outer surface of the enclosure 720. The mounting member 760 can take any desirable size or shape for mounting the enclosure 720 to an apparatus. The apparatus can be a chin rest, stand, tripod mount, optical bench, or other apparatus to maintain the phantom eye 700 in a desirable position for spectral imaging. In some embodiments, the mounting member 760 can be a cavity sized and shaped to allow the enclosure 720 to be friction fit to an apparatus with a correspondingly sized component for being received in the mounting member 760. In some embodiments, the mounting member 760 may be a cavity including one or more grooves within its interior to form a snap fit with a correspondingly sized component of an apparatus. In some embodiments, the mounting member 760 may be a cavity including internal threads for receiving a threaded member, such as a screw, of an apparatus. While embodiments have been discussed wherein the enclosure 720 includes a female mounting member 760 (i.e. the mounting member 760 is sized or shaped to receive one or more components of the apparatus within), it should be appreciated that in some embodiments the mounting member 760 can be a male component formed in or extending from the enclosure 720. For instance, in some embodiments, the mounting member 760 can be a protrusion from the enclosure 720 that is threaded, grooved, or otherwise sized and shaped to be secured within a corresponding female feature of the apparatus. In some embodiments, the apparatus can include an actuator to adjust the position of the phantom eye 700 through its coupling with the mounting member 760.

In some embodiments, the phantom eye 700 can be unitary. That is, the various components of the phantom eye 700, such as the enclosure 720, reflectance standard 730, and ocular media 702, can be fixedly secured to each other. Similarly, it should be appreciated that the various components of the phantom eyes discussed with respect to FIGS. 2-7, including the shells, reflectance standards, and ocular media, can be fixedly secured to each other. In some embodiments, the phantom eye 700 can be modular. That is, various components of the phantom eye 700, such as the enclosure 720, reflectance standard 730, and ocular media 702, can be non-fixedly secured to each other. Similarly, it should be appreciated that the various components of the phantom eyes discussed with respect to FIGS. 2-7, including the shells, reflectance standards, and ocular media, can be non-fixedly secured to each other. In such modular embodiments, the components of the phantom eye 700, or any of the phantom eyes discussed with respect to FIGS. 2-7, can be replaced and/or interchanged as desired. In such modular embodiments, the components of the phantom eye 700, or any of the phantom eyes discussed with respect to FIGS. 2-7, can be connected with, by way of non-limiting example, mounting bobs, threads, adhesive tapes, mounting clamps, or wedges. In some embodiments, the modular connections can be watertight. For instance, in some embodiments, the phantom eye 700, or any of the phantom eyes discussed with respect to FIGS. 2-7, can include one or more rubber O-rings to provide watertight connections between modular parts. Such watertight connections can be used in embodiments where the phantom eye 700 includes water or other liquid within.

In some embodiments, the phantom eye 700 includes one or more tightening fixtures 750. In some embodiments, the tightening fixtures 750 can extend along an anterior surface of the mechanical enclosure 720. In some embodiments, the tightening fixtures 750 can secure the reflectance standard 730 and/or one or more components of the ocular media 702 within the cutout 722. In some embodiments, the tightening fixtures 750 can secure the reflectance standard 730 and/or one or more components of the ocular media 702 within the enclosure 720. The tightening fixtures 750 can be any known or suitable fixation means, such as, but not limited to, screws, sealants, or bolts. In some embodiments, the reflectance standard 730 can be freely positioned within the cutout 722. That is, the reflectance standard 730 may not be fixedly coupled or adhered to the surface 724 of the cutout 722. Similarly, in some embodiments, one or more components of the ocular media 702, such as the sclera 704, may not be fixedly coupled or adhered to an anterior surface of the reflectance standard 730 within the cutout 722. In such embodiments, the tightening fixtures 750 can secure the reflectance standard 730 and one or more components of the ocular media 702 within the cutout 722 and the enclosure 720.

It should be appreciated that the phantom eye 700 can be incorporated in any of the calibration systems discussed with respect to FIGS. 2-7. That is, when incorporated in a calibration system including a lighting assembly, the various components of the phantom eye 700, including the reflectance standard 730 and the ocular media 702 can be positioned relative to each other and the lighting assembly as discussed with respect to any of FIGS. 2-7. Additionally, it should be appreciated that an OEMI-7 eye model is not suitable for the phantom eyes disclosed herein, as an OEMI-7 eye model does not afford access to necessary space to fit a curved reflectance standard of suitable or standard thickness.

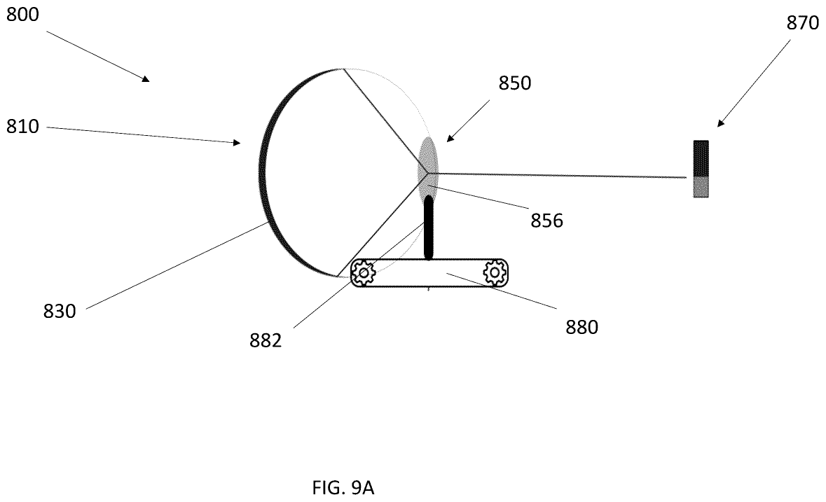

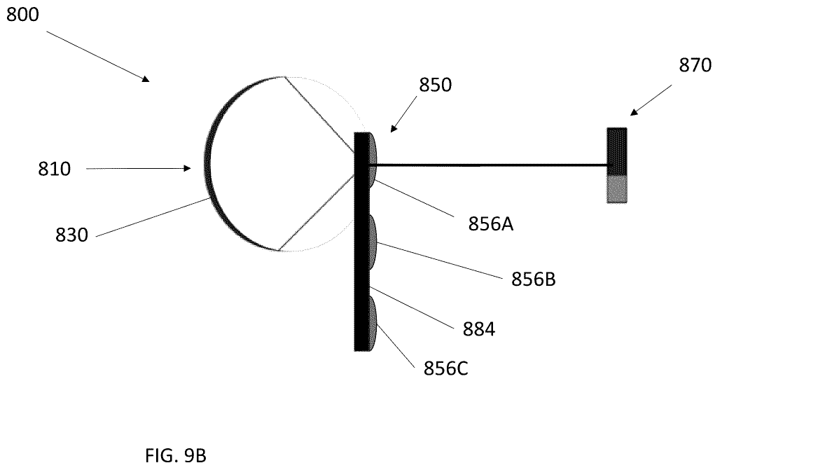

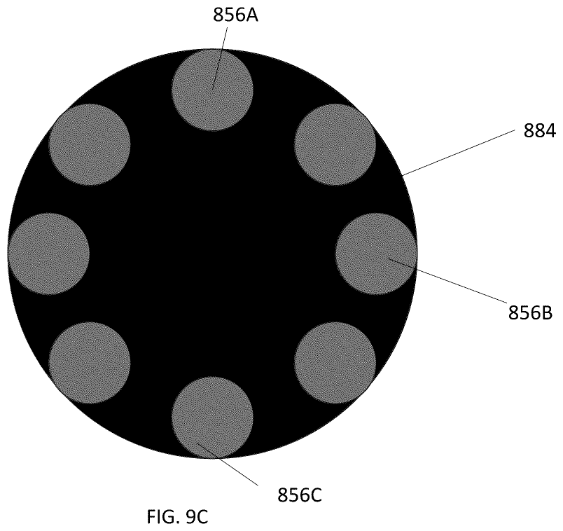

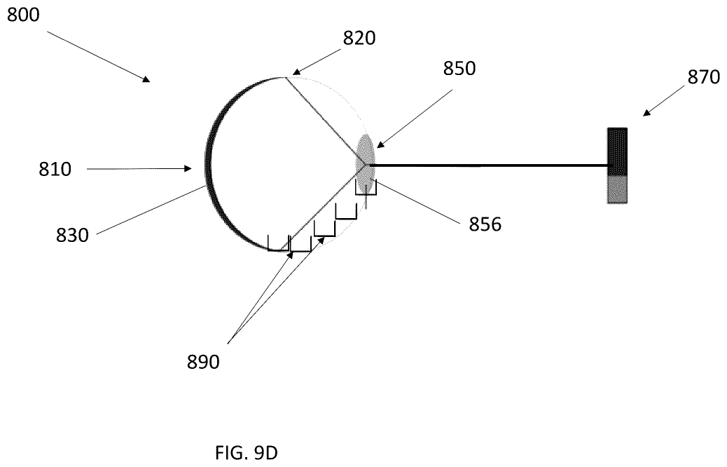

Referring now to FIG. 9A, a calibration system 800 is depicted. The calibration system 800 includes a phantom eye 810, shown in cross section. The phantom eye 810 can resemble any of the phantom eyes discussed with reference to FIGS. 2-8 in all aspects except as noted herein. For instance, the phantom eye 810 can include a reflectance standard 830 and ocular media 850, which can include, at least, an artificial crystalline lens 856. The ocular media 850, including the artificial crystalline lens 856, the reflectance standard 830, and a lighting assembly 870 can be positioned relative each other as discussed with respect to any of FIGS. 2-8. In some embodiments, the artificial crystalline lens 856 is coupled to a track 880. In some embodiments, the track 880 can include one or more movable components, such as a belt positioned on two or more rotating shafts. In some embodiments, the artificial crystalline lens 856 can be coupled to the track 880 by a mount 882. The track 880 can be operated to move the mount 882, and by extension, the artificial crystalline lens 856. Particularly, the track 880 can be operated to move the artificial crystalline lens 856 toward or away from the reflectance standard 830 to achieve a refractive power of interest of the phantom eye 810. For instance, the refractive power of interest can mimic myopic or hyperopic refraction conditions. Operation of the track 880 can be used to mimic different eyes with different shapes and distances between ocular components.

In some embodiments, one or more other ocular components of the ocular media 850, such as an artificial cornea, can be coupled to the track 880 instead of the crystalline lens 856 to adjust the distance of the one or more other ocular components relative the reflectance standard 830. In some embodiments, one or more other ocular components, such as an artificial cornea, can be coupled to the track 880 in addition to the artificial crystalline lens 856 to adjust the distance of the one or more other ocular components, in combination with the artificial crystalline lens 856, relative the reflectance standard 830. In some embodiments, the artificial crystalline lens 856 and the one or more other ocular components are unitarily moved along the track 880, for instance, when one or more components of the ocular media 850 are coupled to each other. In some embodiments, the artificial crystalline lens 856 can be coupled to the track 880 and one or more other ocular components can be coupled to a separate track, such that the artificial crystalline lens 856 and the one or more other ocular components are separately moveable relative the reflectance standard 830. In some embodiments, the reflectance standard 830 can be coupled to the track 880 instead of the artificial crystalline lens 856 or the one or more other ocular components, such that the distance between the reflectance standard 830 and the artificial crystalline lens 856 or one or more other ocular components is adjusted by moving reflectance standard 830 along the track 880 relative the artificial crystalline lens 856 or one or more other ocular components.

Referring now to FIGS. 9B and 9C, the calibration system 800 is depicted including a rotary wheel 884, which is depicted in a frontal view in FIG. 9C, in place of the track 880. In some embodiments, the rotary wheel 884 includes a plurality of artificial crystalline lenses 856A, 856B, 856C positioned thereon. In some embodiments, each of the plurality of artificial crystalline lenses 856A, 856B, 856C can have different refractive powers. In some embodiments, an actuator can drive the rotary wheel 884 to rotate the rotary wheel 884 until a desired one of the plurality of artificial crystalline lenses 856A, 856B, 856C is positioned between the light source of the lighting assembly 870 and the reflectance standard 830 along the optical path of light emitted from the light source of the lighting assembly 870 to the reflectance standard 830. In some embodiments, an actuator can drive the rotary wheel 884 to rotate the rotary wheel 884 until a desired one of the plurality of artificial crystalline lenses 856A, 856B, 856C is positioned between the reflectance standard 830 and the sensor of the lighting assembly 870 along the optical path of light reflected to the sensor from the reflectance standard 830.

In some embodiments, a plurality of other ocular media components, such as artificial corneas can be coupled to the rotary wheel 884 instead of or in addition to the plurality of crystalline lenses 856A, 856B, 856C to allow for selecting a desired artificial cornea of particular ocular properties to be positioned between the light source of the lighting assembly 870 and the reflectance standard 830 along the optical path of light emitted from the light source of the lighting assembly 870 to the reflectance standard 830 or positioned between the reflectance standard 830 and the sensor of the lighting assembly 570 along the optical path of light reflected to the sensor from the reflectance standard 830. In some embodiments, the plurality of artificial crystalline lenses 856A, 856B, 856C and the plurality of other ocular media components are unitarily rotated through on the rotary wheel 884, for instance, when the plurality of other ocular components of the ocular media 850 are respectively coupled to the plurality of artificial crystalline lenses 856A, 856B, 856C. In some embodiments, the plurality of artificial crystalline lenses 856A, 856B, 856C can be coupled to the rotary wheel 884 and a plurality of other ocular components can be coupled to a separate rotary wheel, such that the plurality of artificial crystalline lenses 856A, 856B, 856C and the plurality of other ocular components are separately rotated through. In some embodiments, the plurality of other ocular components can include any of the above-described ocular media components. In some embodiments, the plurality of other ocular components can be optical components including, polarizers films with different orientations, spectral media with different transmittance, spectral flattening filters, or band pass filters, for instance.

Referring now to FIG. 9D, the calibration system 800 is depicted with the phantom eye 810 including a plurality of slots 890. In some embodiments, the plurality of slots 890 can be formed along an inner surface of a shell 820 of the phantom eye 810. In some embodiments, each of the plurality of slots 890 can be positioned a different distance from the reflectance standard 830. In some embodiments, the shell 820 of the phantom eye 810 can include one or more openings to allow a user access to an interior of the shell 820. A user can selectively place the artificial crystalline lens 856 in a desired one of the plurality of slots 890 such that the artificial crystalline lens 856 is positioned a desired distance from the reflectance standard 830 and a desired refractive power is achieved. In some embodiments, one or more other ocular media components, such as an artificial cornea can be selectively placed in one of the plurality of slots 890 instead of or in addition to the artificial crystalline lens 856 to selectively adjust the distance of the one or more other ocular components relative the reflectance standard 830. In some embodiments, the artificial crystalline lens 856 and the one or more other ocular media or optical components are unitarily placed in one of the plurality of slots 890, for instance, when one or more components of the ocular media 850 are coupled to each other.

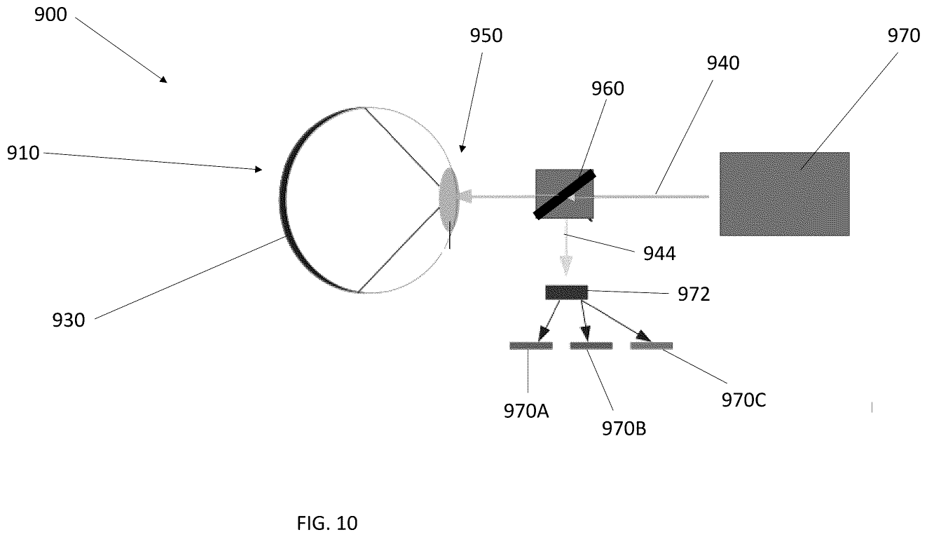

Referring now to FIG. 10, a calibration system 900 is depicted. The calibration system 900 includes a phantom eye 910, shown in cross section. The phantom eye 910 can resemble any of the phantom eyes discussed with reference to FIGS. 2-9D in all aspects except as noted herein. For instance, the phantom eye 910 can include a reflectance standard 930, ocular media 950, and a lighting assembly 970, which may include a light source and a sensor. The ocular media 950, the reflectance standard 930, and the lighting assembly 970 can be positioned relative each other as discussed with respect to any of FIGS. 2-9D. The calibration system 900 can further include a beam splitter 960, or beam combiner, positioned between the lighting assembly 970 and the phantom eye 910 along the optical path of light 940 emitted by a light source of the lighting assembly 970 to the reflectance standard 930. The beam splitter 960 can have known spectral characteristics to direct n % of the incoming irradiance from the optical path of light 940 as a split beam 944 for optical metrology purposes. In some embodiments, the beam splitter 960 can direct light to one or more optical fibers so that commercial light meters can be used to measure characteristics of the split beam 944. The beam splitter 960 can have known spectral characteristics to inject external sources such as LED, lasers, or any other kind of light source for metrology and calibration purposes.

Any number of sensors or measurement devices can be used, along with the beam splitter 960, as desirable. In some embodiments, a fiber end holder can be selected based on particular user needs. For instance, in some embodiments, multiple fiber end holders 970A, 970B, and 970C can be used to optically couple one or optical fibers for input or output of one or more optical sensors or light sources with the split beam 944, such that the one or more optical sensors can measure one or more characteristics of the split beam 944, which in turn may be attributable to the light emitted by the lighting assembly 970 along the optical path of light 940. In some embodiments, each of the fiber end holders 970A, 970B, and 970C can be situated in a mechanical holder 972, which can include a fiber splitter such that the split beam 944 can be simultaneously measured by the one or more optical sensors coupled to the fiber end holders 970A, 970B, and 970C. Non-limiting example measurement devices or sensors optically coupled to the split beam 944 include a spectrophotometer (e.g., high-resolution HR4 Pro) for measuring spectral shape, a power meter (e.g., ILT5000, Thorlabs meters) for measuring absolute or relative total irradiance, which may then be used to correct a spectrophotometer measurement, a wavefront sensor (which may be used for spectral Adaptive Optics Scanning Laser Ophthalmoscopy (AOSLO)), one or more sensors to measure the polarization state of the light (e.g., a polarization state analyzer (PSA) or polarimeter), a photodiode for irradiance, and/or a low-cost multi-spectral sensor (e.g., AMS AS7341), which may be a fixed sensor added to a custom spectral camera setup. In some embodiments, the beam splitter 960, and related optical sensors, can be used to measure the spectral irradiance of light emitted by the lighting assembly 970 before it enters the phantom eye 910 and is reflected back to the sensor of the lighting assembly 970. For instance, the beam splitter 960 and associated sensors may be used to determine if the light emitted by the lighting assembly 970 is stable over time.

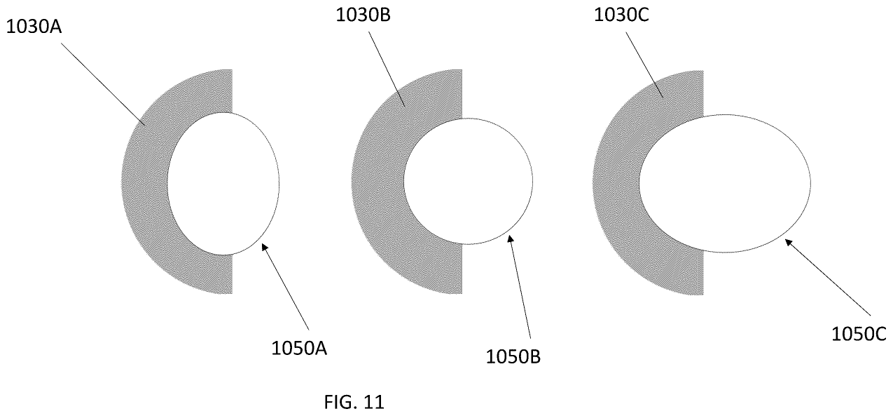

The components of the phantom eyes discussed above with respect to FIGS. 2-10, can be particularly shaped to mimic a biological eye, regardless of whether the phantom eye is unitary or modular. More specifically, the phantom eyes, and their various sub-components, including the reflectance standard and ocular media, may not be single-sized. For instance, one or more components of the above-described phantom eyes can be particularly shaped to take into account, by way of non-limiting example, heavy myopia/hyperopia, the axial length increase with age/myopia, hyperopia, and/or emmetropia. Therefore, the phantom eye can be able to simulate different refractions with the ocular media and reflectance standard of custom curvature. FIG. 11 depicts three combinations of reflectance standards 1030A, 1030B, and 1030C with ocular media 1050A, 1050B, and 1050C, respectively, showing variations of curvatures in phantom eye components. In some embodiments, the above-described phantom eyes can include shape-memory alloys, piezoelectric actuators, or other selectively deformable components coupled to the reflectance standards, ocular media, or other components of the above-described phantom eyes to selectively adjust the curvature of the reflectance standards, ocular media, or other components.

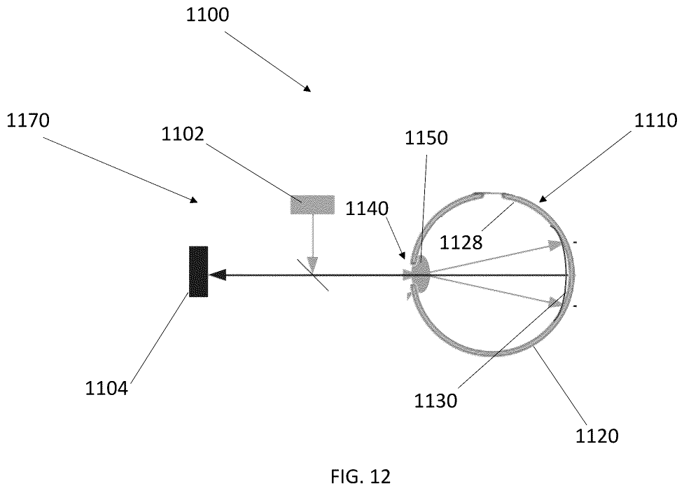

Referring now to FIG. 12, a calibration system 1100 is depicted. The calibration system 1100 can resemble any of the above-described calibration systems, except as noted herein. In some embodiments, the calibration system 1100 includes a phantom eye 1110, shown in cross section, and a lighting assembly 1170 including a light source 1102 and a sensor 1104. In some embodiments, the phantom eye 1110 includes an integrating sphere 1120 forming the shell of the phantom eye 1110. In some embodiments, the integrating sphere 1120 can include a curved inner surface 1128. In some embodiments, the curved inner surface 1128 can be coated with a curved reflectance standard 1130. In some embodiments, the reflectance standard 1130 can be coated on a posterior portion of the curved inner surface 1128 of the integrating sphere 1120. In some embodiments, the integrating sphere 1120 can include an opening 1140 in an anterior portion of the integrating sphere 1120, allowing light into and out of the integrating sphere 1120. In some embodiments, ocular media 1150 can be positioned in the opening 1140 of the integrating sphere 1120. In some embodiments, ocular media 1150 can be positioned in the opening 1140 of the integrating sphere 1120 similar to the attachments previously described between the ocular media and shells of the above-described phantom eyes. In some embodiments, the ocular media 1150 can include any or all of the ocular media components described above with respect to FIGS. 2-11. In some embodiments, the ocular media 1150 is positioned between the light source 1102 of the lighting assembly 1170 and the reflectance standard 1130 along the optical path of light emitted from the light source 1102 to the reflectance standard 1130. In some embodiments, the ocular media 1150 is positioned between the sensor 1104 of the lighting assembly 1170 and the reflectance standard 1130 along the optical path of light reflected to the sensor 1104 from the reflectance standard 1130. In some embodiments, the ocular media 1150 is positioned anterior the reflectance standard 1130.

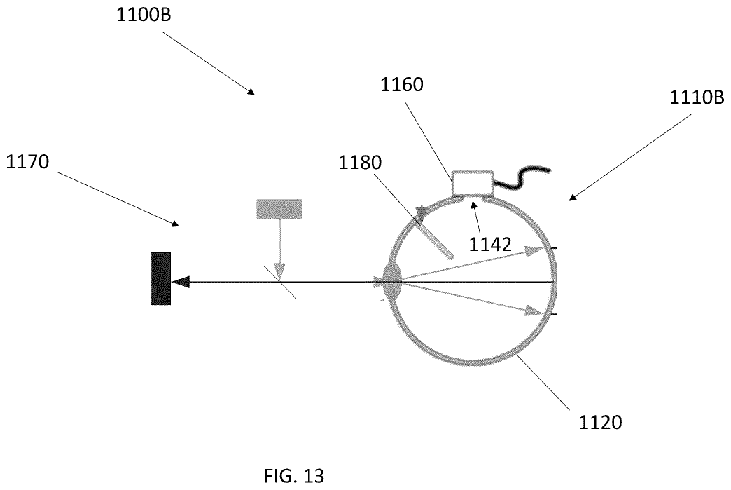

Reference is now made to FIG. 12 in conjunction with FIG. 13, which depicts another embodiment of a phantom eye 1110B of the calibrations system 1100. The phantom eye 1110B can resemble the phantom eye 1110 described with respect to FIG. 12 in all aspects, except as noted herein. Particularly, in some embodiments, the phantom eye 1110B includes a second opening 1142 in the integrating sphere 1120. In some embodiments, the second opening 1142 in the integrating sphere 1120 allows for positioning one or more additional light sensors 1160 on or along the integrating sphere 1120 and having a sensing area directed towards the interior of the integrating sphere 1120. The one or more additional light sensors 1160 can provide feedback on the power, intensity, wavelength, polarization, or other characteristics of the light reflected by the reflectance standard 1130 coated on the inner surface 1128. Merely as an example, the one or more additional light sensors 1160 can include a power meter or a point spectrometer to measure irradiance or spectral irradiance of the light reflected to the sensor 1104. In some embodiments, the one or more additional light sensors 1160 can be a photodiode or spectrophotometer. In practice, this may allow the use of high spectral resolution point spectrometers as the one or more additional light sensors 1160 simultaneously with lower resolution spectral cameras as the sensor 1104. In some embodiments, the one or more additional light sensors 1160 can provide feedback on whether the phantom eye 1110B is spectrally functioning as designed or expected. For instance, the one or more additional light sensors 1160 can provide an indication that the interior of the phantom eye 1110B is contaminated or damaged. In some embodiments, the spectral sensitivity of the sensor 1104 can be characterized if using a controllable light source 1102. For instance, if using a polychromatic incoherent light source and a monochromator (or supercontinuum laser with acousto-optic tunable filters, liquid crystal tunable filter, filter wheel), a user can scan the wavelengths and record the response. In some embodiments, the phantom eye 1110B can include a baffle 1180. The baffle 1180 can block the one or more additional light sensors 1160 from the light emitted from the light source 1102 to ensure the one or more additional light sensors 1160 measure light reflected by the reflectance standard 1130. In some embodiments, the second opening 1142 can be selectively sealed with a cap coated in the reflectance standard 1130. Therefore, the one or more additional light sensors 1160 may be selectively used in the calibrations system 1100B depending on whether the second opening 1142 is sealed with the cap, and the one or more additional light sensors 1160 is, therefore, covered and blocked from receiving light. In some embodiments, the second opening 1142 can be coupled with a fiber and a spectrometer. In some embodiments, the baffle 1180 can be coupled with a fiber and a spectrometer or used to simulate intra-corneal illumination conditions.

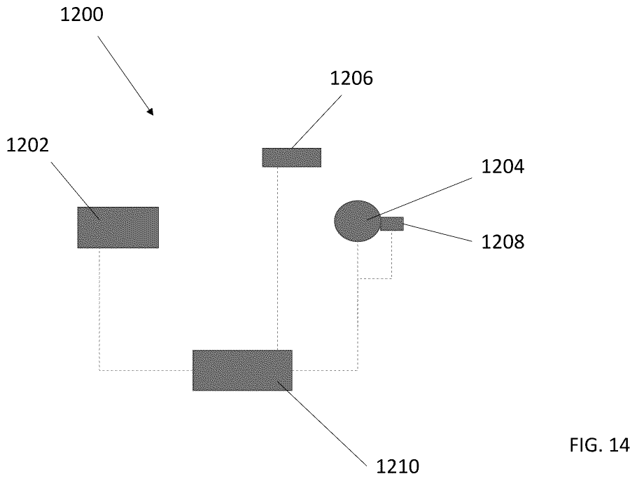

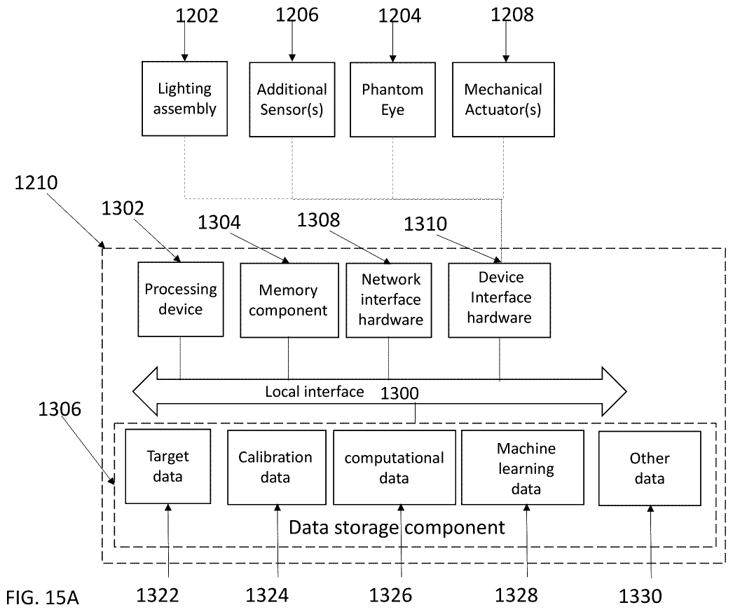

Referring now to FIG. 14, a schematic calibration system 1200 is depicted. The calibration system 1200 can be any of the above-described calibration systems. In some embodiments, the calibration system 1200 generally includes a lighting assembly 1202, which can take the form and function of any of the above-described lighting assemblies and general includes a light source and a sensor, and a phantom eye 1204, which can take the form and function of any of the above-described phantom eyes. In some embodiments, the calibration system 1200 can include one or more additional sensors 1206, such as the one or more additional light sensors 1160 discussed with respect to FIG. 13 or the one or more optical sensors coupled to the end holders 970A, 970B, and 970C discussed with respect to FIG. 10. In some embodiments, the calibration system 1200 includes one or more mechanical actuators 1208 coupled to one or more components of the phantom eye 1204, such as the track 880 discussed with respect to FIG. 9A, the rotary wheel 884 discussed with respect to FIGS. 9B and 9C, or the actuator coupled with the mounting member 760 as discussed with respect to FIG. 8. In some embodiments, the calibration system 1200 includes an electronic control unit 1210. In some embodiments, the lighting assembly 1202, phantom eye 1204, one or more additional sensors 1206, or one or more mechanical actuators 1208 are communicatively coupled with the electronic control unit 1210, as depicted by the dashed lines of FIG. 14, such that the electronic control unit 1210 directs operation of the lighting assembly 1202, phantom eye 1204, one or more additional sensors 1206, or one or more mechanical actuators 1208. As used herein, the term “communicatively coupled” generally refers to any link in a manner that facilitates communications. As such, “communicatively coupled” includes both wireless and wired communications, including those wireless and wired communications now known or later developed. As the electronic control unit 1210 is communicatively coupled to the lighting assembly 1202, phantom eye 1204, one or more additional sensors 1206, or one or more mechanical actuators 1208, one or more signals, data, or the like may be transmitted between the electronic control unit 1210 and the lighting assembly 1202, phantom eye 1204, one or more additional sensors 1206, or one or more mechanical actuators 1208. Therefore, the electronic control unit 1210 is generally a device that is communicatively coupled to one or more components of the calibration system 1200 and is particularly arranged and configured to transmit, receive, or read signals or data to/from the one or more components of the calibration system 1200.

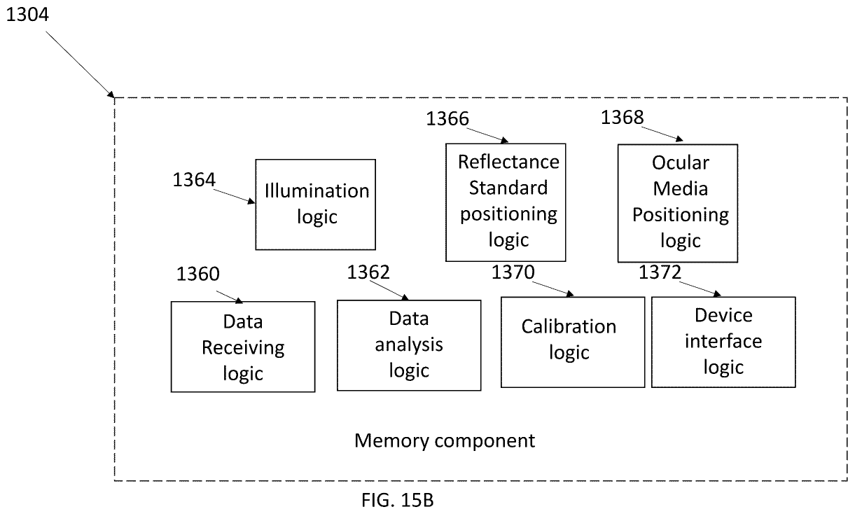

In some embodiments, the electronic control unit 1210 can be located proximate to or distant from (including geographically remote from) the other components of the calibration system 1200. For instance, in some embodiments, the electronic control unit 1210, including a processor, can be implemented in a server or cloud computing system that is located geographically remote from the other components of the calibrations system 1200. In some embodiments, the electronic control unit 1210 can be integrated with, such as in a same housing as, the sensor of the lighting assembly 1202. Additional details regarding the electronic control unit 1210 will be discussed herein with respect to FIGS. 15A and 15B.

Referring now to FIG. 14 and FIG. 15A, the various internal components of the electronic control unit 1210 are shown. Particularly, FIG. 15A depicts various system components for collecting a spectral calibration reference measurement from a phantom eye and using the reference measurement to calibrate, or correct, a spectral image of a target biological eye of interest. As illustrated in FIG. 15A, the electronic control unit 1210 can include one or more processing devices 1302, a non-transitory memory component 1304, network interface hardware 1308, device interface hardware 1310, and a data storage component 1306. A local interface 1300, such as a bus or the like, can interconnect the various components.

The one or more processing devices 1302, such as a computer processing unit (CPU), can be the central processing unit of the electronic control unit 1210, performing calculations and logic operations to execute a program. The one or more processing devices 1302, alone or in conjunction with the other components, are illustrative processing devices, computing devices, processors, or combinations thereof. The one or more processing devices 1302 can include any processing component configured to receive and execute instructions (such as from the data storage component 1306 and/or the memory component 1304).

The memory component 1304 can be configured as a volatile and/or a non-volatile computer-readable medium and, as such, may include random access memory (including SRAM, DRAM, and/or other types of random access memory), read only memory (ROM), flash memory, registers, compact discs (CD), digital versatile discs (DVD), and/or other types of storage components. The memory component 1304 can include one or more programming instructions thereon that, when executed by the one or more processing devices 1302, cause the one or more processing devices 1302 to complete various processes.

Still referring to FIG. 15A, the programming instructions stored on the memory component 1304 can be embodied as a plurality of software logic modules, where each logic module provides programming instructions for completing one or more tasks. FIG. 15B depicts the various modules of the memory component 1304 of FIG. 15A according to various embodiments.

As shown in FIG. 15B, the memory component 1304 includes a plurality of logic modules. Each of the logic modules shown in FIG. 15B can be embodied as a computer program, firmware, or hardware, as an example. Illustrative examples of logic modules present in the memory component 1304 include, but are not limited to, data receiving logic 1360, data analysis logic 1362, illumination logic 1364, reflectance standard positioning logic 1366, ocular media positioning logic 1368, calibration logic 1370, and device interface logic 1372.

Referring to FIGS. 14, 15A, and 15B, in some embodiments the data receiving logic 1360 includes one or more programming instructions for receiving data from the sensor of the lighting assembly 1202. That is, the data receiving logic 1360 can cause a connection between the device interface hardware 1310 and the sensor (e.g., the sensor 204 (FIG. 2)) of the lighting assembly 1202 such that data transmitted by the sensor is received by the electronic control unit 1210. Further, the data transmitted by the sensor of the lighting assembly 1202 can be stored (e.g., within the data storage component 1306). In some embodiments, the data receiving logic 1360 can further include one or more programming instructions for receiving data from the one or more additional sensors 1206. That is, the data receiving logic 1360 can cause a connection between the device interface hardware 1310 and the one or more additional sensors 1206 such that data transmitted by the one or more additional sensors 1206 is received by the electronic control unit 1210. Further, the data transmitted by the one or more additional sensors 1206 can be stored (e.g., within the data storage component 1306).

In some embodiments, the data analysis logic 1362 includes one or more programming instructions for analyzing data received from the sensor of the lighting assembly 1202 or the one or more additional sensors 1206. For instance, the data analysis logic 1362 can contain programming for analyzing the data collected by the sensor of the lighting assembly 1202 and generating a reference image of the phantom eye 1204 from the data collected. The data analysis logic 1362 can further include programming instructions for analyzing data collected by the one or more additional sensors 1206 and determining one or more characteristics or parameters of the light emitted from the light source of the lighting assembly 1202 or reflected to the sensor of the lighting assembly, for instance, based on the data collected.

Still referring to FIGS. 14, 15A, and 15B, in some embodiments the illumination logic 1364 includes one or more programming instructions for directing the light source of the lighting assembly 1202 to emit light to the phantom eye 1204. The illumination logic 1364 can include one or more programming instructions for controlling the positioning of the light source of the lighting assembly 1202 and the direction in which light is emitted from the light source of the lighting assembly 1202. The illumination logic 1364 can include one or more programming instructions for controlling one or more parameters of the light source of the lighting assembly 1202, based on the data collected by the one or more additional sensors 1206, for instance.

In some embodiments, the reflectance standard positioning logic 1366 includes one or more programming instructions for adjusting the shape or position of the reflectance standard (e.g., the reflectance standard 230 depicted in FIG. 2) of the phantom eye 1204. In some embodiments, the reflectance standard positioning logic 1366 can include one or more programming instructions for operating one or more actuators of the phantom eye 1204. For instance, the reflectance standard positioning logic 1366 can include one or more programming instructions for operating the actuator 740 (FIG. 8) coupled to the reflectance standard 730 (FIG. 8) to adjust the positioning of the reflectance standard 730 (FIG. 8). In some embodiments, the reflectance standard positioning logic 1366 can include one or more programming instructions for actuating one or more selectively deformable components, such as shape-memory alloys or piezoelectric actuators, coupled to the reflectance standard of the phantom eye 1204, as discussed with respect to FIG. 11, to selectively adjust the curvature and shape of the reflectance standard. In some embodiments, the reflectance standard positioning logic 1366 can include one or more programming instructions for operating the one or more mechanical actuators 1208 coupled to the phantom eye 1204. For instance, in some embodiments, the reflectance standard positioning logic 1366 may include one or more programming instructions for operating a track 880 (FIG. 9A) to which the reflectance standard is coupled, as discussed with respect to FIG. 9A.

Still referring to FIGS. 14, 15A, and 15B, in some embodiments the ocular media positioning logic 1368 can include one or more programming instructions for adjusting the position of one or more components of the ocular media (e.g., the ocular media 250 depicted in FIG. 2) of the phantom eye 1204. For instance, in some embodiments, the ocular media positioning logic 1368 can include one or more programming instructions for operating the one or more mechanical actuators 1208 coupled to the phantom eye 1204. For instance, in some embodiments, the ocular media positioning logic 1368 can include one or more programming instructions for operating the track 880 (FIG. 9A) to which the one or more components of the ocular media is coupled, as discussed with respect to FIG. 9A. In some embodiments, the ocular media positioning logic 1368 can include one or more programming instructions for operating the rotary wheel 884 (FIGS. 9B and 9C) to which one or more components of the ocular media is coupled, as discussed with respect to FIGS. 9B and 9C.

In some embodiments, the calibration logic 1370 can include one or more programming instructions for correcting or adjusting a spectral retinal image of a biological target eye. In some embodiments, the calibration logic 1370 can include one or more programming instructions to perform one or more mathematical operations to correct or adjust a spectral retinal image of a biological eye with the reference or calibration image generated of the phantom eye 1204. Particularly, in some embodiments, the calibration logic 1370 can include one or more programming instructions to correct or adjust a spectral retinal image of a biological eye with the reference or calibration image generated of the phantom eye 1204 by dividing pixel values of the spectral retinal image of the biological eye by pixel values of the reference or calibration image of the phantom eye 1204.

Still referring to FIGS. 14, 15A, and 15B, in some embodiments, the device interface logic 1372 includes one or more programming instructions for establishing communicative connections with the various devices or components of the calibration system 1200. For example, the device interface logic 1372 can include programming instructions usable to establish connections with the lighting assembly 1202, the phantom eye 1204, the one or more additional sensors 1206, or the mechanical actuators 1208 in various embodiments.

Referring again to FIG. 15A, the network interface hardware 1308 can include any wired or wireless networking hardware, such as a modem, LAN port, wireless fidelity (Wi-Fi) card, WiMax card, mobile communications hardware, or other hardware for communicating with other networks or devices. For example, the network interface hardware 1308 can be used to facilitate communication between external storage devices, user computing devices, server computing devices, external control devices, or the like via a network, such as, for example, a local network, the Internet, etc.

Referring to FIGS. 14 and 15A, in some embodiments, the device interface hardware 1310 can communicate information between the local interface 1300 and one or more components of the calibration system 1200. For example, the device interface hardware 1310 can act as an interface between the local interface 1300 and the lighting assembly 1202, the phantom eye 1204, the one or more additional sensors 1206, or the mechanical actuators 1208. The device interface hardware 1310 can transmit or receive signals or data to/from the sensor of the lighting assembly 1202 or the one or more additional sensors 1206. The device interface hardware 1310 can transmit control signals to the light source of the lighting assembly 1202, the sensor of the lighting assembly 1202, the phantom eye 1204, the one or more additional sensors 1206, or the mechanical actuators 1208.

Still referring to FIGS. 14 and 15A, the data storage component 1306, which can generally be a storage medium, can contain one or more data repositories for storing data that is received or generated. The data storage component 1306 can be any physical storage medium, including, but not limited to, a hard disk drive (HDD), memory, removable storage, and/or the like. While the data storage component 1306 is depicted as a local device, it should be understood that the data storage component 1306 can be a remote storage device, such as, for example, a server computing device, cloud-based storage device, or the like. Illustrative data that can be contained within the data storage component 1306 includes, but is not limited to, target data 1322, calibration data 1324, computational data 1326, machine learning data 1328, and other data 1330.

The target data 1322 can generally be data that is used by the electronic control unit 1210 to position the phantom eye 1204, the reflectance standard of the phantom eye 1204, or the ocular media of the phantom eye 1204 as desired. For instance, the target data 1322 can include data related to one or more biological eyes, including the optical power, shape, and positioning of various portions of the one or more biological eyes. In some embodiments, the one or more biological eyes can be a target biological eye of which a spectral retinal image is gathered of. The calibration data 1324 can generally be data that is used by the electronic control unit 1210 to match one or more phantom eye configurations (e.g., the positioning the phantom eye 1204, the positioning of the reflectance standard of the phantom eye 1204, and/or the positioning of the ocular media of the phantom eye 1204) with the data related to one or more biological eyes. The calibration data 1324 can generally include data that is used by the electronic control unit 1210 to match a spectral image of a particular configuration of a phantom eye with data related to a spectral image of one or more biological eyes. For instance, the calibration data can include a repository of spectral images of phantom eyes having different configurations, from which a particular spectral image is selected for correction of a spectral image of a biological eye of interest, where the configuration of the phantom eye of which the spectral image is selected most closely mimics the optics of the biological eye spectrally imaged. The computational data 1326 can generally be data used by the electronic control unit 1210 to perform one or more mathematical operations to correct or adjust a spectral retinal image of a biological eye with the reference or calibration image generated of the phantom eye 1204. The machine learning data 1328 can generally be data that is generated as a result of one or more machine learning processes used to improve the matching of a phantom eye configuration with a biological eye or the corrections of a spectral retinal image of a biological eye with the reference or calibration image generated of the phantom eye 1204, for instance. The other data 1330 can generally be any other data that is usable for the purposes of configuring the phantom eye 1204, correcting a spectral image of a biological eye, or the like, as described herein.

It should be understood that the components illustrated in FIGS. 15A and 15B are merely illustrative and are not intended to limit the scope of this disclosure. More specifically, while the components in FIGS. 15A and 15B are illustrated as residing within the electronic control unit 1210, this is a nonlimiting example. In some embodiments, one or more of the components can reside external to the electronic control unit 1210.

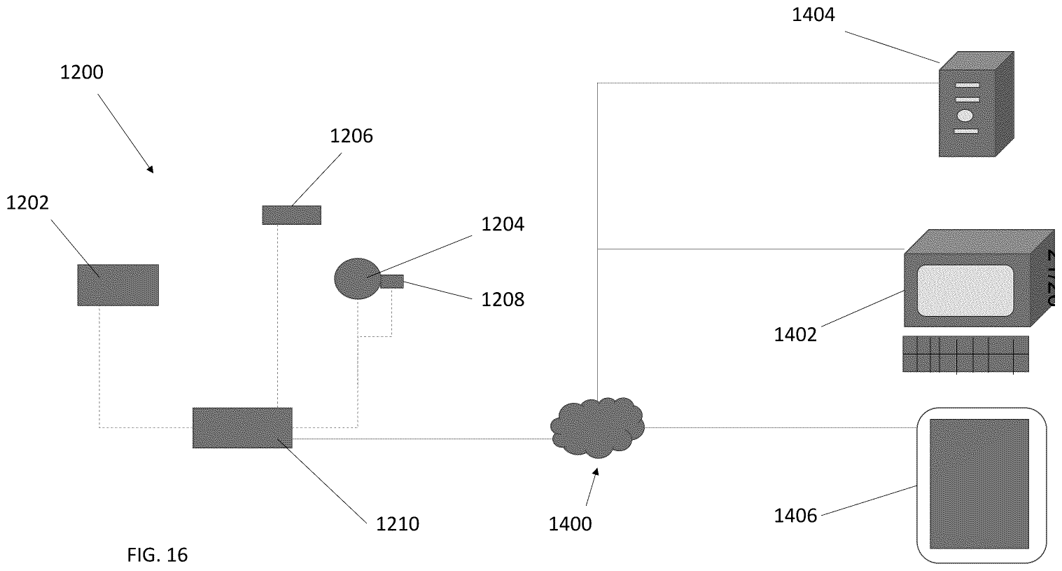

Referring now to FIG. 16 in conjunction with FIG. 14, an illustrative control network 1400 is depicted. As illustrated in FIG. 16, the control network 1400 can include a wide area network (WAN), such as the Internet, a local area network (LAN), a mobile communications network, a public service telephone network (PSTN), a personal area network (PAN), a metropolitan area network (MAN), a virtual private network (VPN), or another network. The control network 1400 can generally be configured to electronically connect one or more systems or devices, such as, for example, computing devices, servers, electronic devices, calibration systems, or components of any of the foregoing. Illustrative systems or devices can include, but are not limited to, a user computing device 1402, a database server 1404, an electronic device 1406, or the electronic control unit 1210 of the calibration system 1200.

Still referring to FIGS. 14 and 16, the user computing device 1402 can generally be used as an interface between a user and the other components connected to the control network 1400. Thus, the user computing device 1402 can be used to perform one or more user-facing functions, such as receiving one or more inputs from a user or providing information to the user. Accordingly, the user computing device 1402 can include at least a display or input hardware. In the event that any of the other devices connected to the control network 1400 (e.g., the database server 1404, the electronic device 1406, or the electronic control unit 1210) requires oversight, updating, or correction, the user computing device 1402 can be configured to provide the desired oversight, updating, or correction. The user computing device 1402 can also be used to input data that is usable to determine a particular phantom eye configuration to be used, a desired light source power, or the like. That is, a user can input information via the user computing device 1402 to control various parameters of the calibration system 1200.

The database server 1404 can generally be a repository of data that is used for the purposes of adjusting a phantom eye configuration or a light source, as described herein. That is, the database server 1404 can contain one or more storage devices for storing data pertaining to information received from the lighting assembly 1202, one or more additional sensors 1206, a biological eye under study, any generated calculations, or the like. In some embodiments, the database server 1404 can contain information therein that mirrors the information stored in the data storage component 1306 (FIG. 15A) or can be used as an alternative to the data storage component 1306 (FIG. 15A), such as an offsite data repository. The database server 1404 can be accessible by one or more other devices or systems coupled to the control network 1400 and can provide the data as needed.

The electronic device 1406 can generally be any device that contains hardware that is operable to be used as an interface between a user and the other components of the control network 1400. Thus, the electronic device 1406 can be used to perform one or more user-facing functions, such as, for example, receiving data one or more external components, displaying information to a user, receiving one or more user inputs, transmitting signals corresponding to the one or more user inputs, or the like. For instance, the electronic device 1406 can present an alert to a user if, based on the data analyzed from the one or more additional sensors 1206, it is determined that the phantom eye 1204 is contaminated or damaged. While FIG. 16 depicts the electronic device 1406 as a smart phone, it should be understood that this is a nonlimiting example. That is, the electronic device 1406 can be any mobile phone, a tablet computing device, a personal computing device (e.g., a personal computer), and/or the like.

It should be understood that while the user computing device 1402 is depicted as a personal computer, the database server 1404 is depicted as a server, and the electronic device 1406 is depicted as a mobile device, these are nonlimiting examples. In some embodiments, any type of computing device (e.g., mobile computing device, personal computer, server, cloud-based network of devices, etc.) or specialized electronic device can be used for any of these components. Additionally, while each of these computing devices is illustrated in FIG. 16 as a single piece of hardware, this is also merely an example. Each of the user computing device 1402, the database server 1404, and the electronic device 1406 can represent a plurality of computers, servers, databases, components, and/or the like.

While FIG. 16 depicts the various systems or components communicatively coupled to one another via the control network 1400, this is merely illustrative. In some embodiments, various components can be communicatively coupled to one another via a direct connection. In some embodiments, various components can be integrated into a single device.

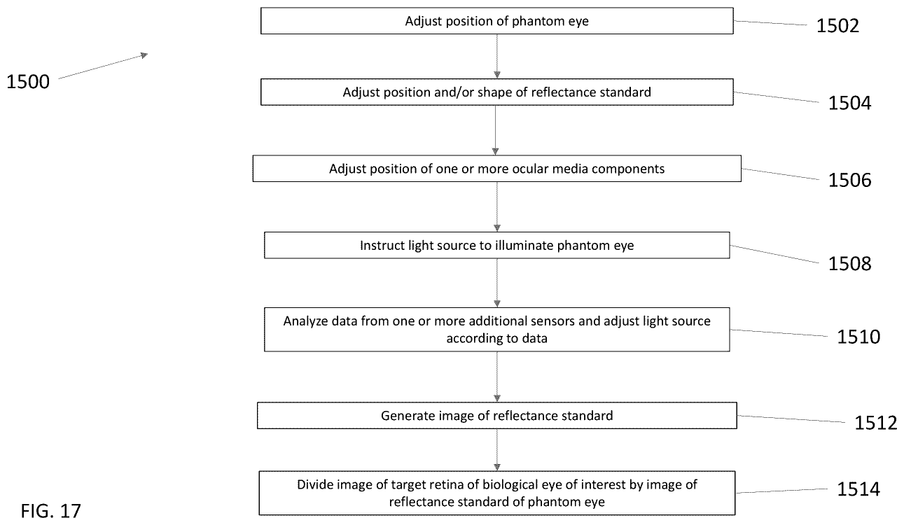

The various embodiments depicted in FIGS. 1-16 should now generally be understood. The operation of the above-described calibration systems will now be discussed with reference to the illustrative method 1500 depicted in FIG. 17. Reference will, also, be made to the calibration system 200 discussed with respect to FIGS. 2 and 3. At a step 1502 of the method 1500, the position of the phantom eye 210 can be adjusted as desired. For instance, in embodiments where an actuator is coupled to the mounting member 760 (FIG. 8), the actuator can be controlled to move the phantom eye 210 into a desirable position relative the lighting assembly 270 for imaging.

At a step 1504 of the method 1500, the position or shape of the reflectance standard 230 can be adjusted. In some embodiments, where the reflectance standard 230 is interchangeable, a user can manually insert a reflectance standard 230 of desired shape within the phantom eye 210. In some embodiments, where the reflectance standard 230 is coupled to an actuator 740 (FIG. 8) within the phantom eye 210, the actuator 740 (FIG. 8) can be controlled to adjust the positioning of the reflectance standard 230 within the phantom eye 210. In some embodiments, where the reflectance standard 230 is coupled to the track 880 (FIG. 9A), the track 880 (FIG. 9A) can be controlled such that the distance between the reflectance standard 230 and the crystalline lens 256 or one or more other ocular media 250 components is adjusted. In some embodiments, where the phantom eye 210 includes shape-memory alloys, piezoelectric actuators, or other selectively deformable components coupled to the reflectance standard 230, the deformable components can be controlled to selectively adjust the curvature of the reflectance standard 230.

At a step 1506 of the method 1500, the position or shape of one or more ocular media 250 components can be adjusted. For instance, in some embodiments where the one or more ocular media 250 components are coupled to the track 880 (FIG. 9A), the track 880 (FIG. 9A) can be controlled such that the distance between the one or more ocular media 250 components and the reflectance standard 230 is adjusted. In some embodiments, where one or more ocular media 250 components are coupled to the rotary wheel 884 (FIG. 9B), the rotary wheel 884 (FIG. 9B) can be driven such that ocular media 250 components of desired characteristics are positioned between the light source 202 and the reflectance standard 230 along the optical path of light emitted from the light source 202 to the reflectance standard 230 or positioned between the reflectance standard 230 and the sensor 204 along the optical path of light reflected to the sensor 204 from the reflectance standard 230. In some embodiments, the position of the one or more ocular media 250 components can be manually adjusted. For instance, in some embodiments, a user can selectively place one or more ocular media 250 components in one of a plurality of slots 890 (FIG. 9D) of the phantom eye 210. In some embodiments, where the phantom eye 210 includes shape-memory alloys, piezoelectric actuators, or other selectively deformable components coupled to one or more ocular media 250 components, the deformable components can be controlled to selectively adjust the curvature of the one or more ocular media 250 components.

At a step 1508 of the method 1500, the light source 202 of the lighting assembly 270 can be instructed to illuminate the phantom eye 210, and particularly the reflectance standard 230 of the phantom eye 210. The light source 202 can be instructed to illuminate the reflectance standard 230 with light of a desired power, intensity, spectrum, and/or wavelength. The illumination path of the phantom eye 210 can be nonscleral or axial. the That is, the use of the phantom eyes discussed above is not limited to coaxial fundus illumination or the so-called Maxwellian Illumination. The phantom eyes discussed above can be used with any type of fundus illumination, such as by non-limiting example, transscleral illumination, trans-pars-planar illumination, or transcranial illumination.

At a step 1510 of the method 1500, data from one or more additional sensors 1206 (FIG. 14) can be analyzed. For instance, in some embodiments, data can be collected from the one or more additional light sensors 1160 (FIG. 13) or the one or more optical sensors coupled to the end holders 970A, 970B, and 970C (FIG. 10). The data collected from the one or more additional sensors 1206 (FIG. 14) can be analyzed to determine one or more characteristics or parameters of the light emitted from the light source 202 to the reflectance standard 230. Based on the analysis of the data, one or more parameters of the light source 202 can be adjusted such that light of a desired power, intensity, spectrum, and/or wavelength is directed to the reflectance standard 230. Based on this adjustment, the method 1500 can restart from step 1502, restart from a different step, or proceed to a step 1512.

At a step 1512 of the method 1500, a spectral image of the reflectance standard 230 can be generated. In some embodiments, the sensor 204 of the lighting assembly 270 collects light reflected by the reflectance standard 230. The sensor 204 can be configured to generate a spectral image of the reflectance standard 230 based on the light reflected by the reflectance standard 230 to the sensor 204. The spectral image of the reflectance standard 230 can be a reference image to correct a spectral image of a biological eye.

At a step 1514 of the method 1500, a spectral image of a retina or fundus of a biological eye of interest may be corrected using the spectral image of the reflectance standard 230. Particularly, in some embodiments, the spectral image of the retina of the biological eye may be divided by the spectral image of the reflectance standard 230 to correct or remove artifacts in the spectral image of the retina of the biological eye. The corrected spectral image of the retina of the biological eye may then be more accurately analyzed for disease diagnosis. In some embodiments, prior to correcting the spectral image of the retina with the spectral image of the reflectance standard 230, one or both of the spectral image of the retina or the spectral image of the reflectance standard 230 can be corrected with one or more other images. The one or more other images can be a dark, a background, a backreflection, or a straylight image, for instance. These one or more other images can be related to the optical systems or residual artifacts, aberrations, or misalignments of the phantom eye.

It should be appreciated that, in some embodiments, the phantom eye 210 can be particularly configured to mimic a known biological eye of interest. For instance, a user may first analyze a biological eye of interest that is under study to determine physiology and structure of the biological eye, including the shape and positioning of the retina and shape, positioning, or optical properties (e.g. refractive power) of the ocular media of the biological eye. Based on this analysis, the steps 1504 and 1506 of the method 1500 can be conducted to arrange the phantom eye 210 to most closely mimic the biological eye under study. In other embodiments, a database of reference images of phantom eyes can be generated. For instance, the steps 1504-1512 of the method 1500 can be carried out for a plurality of different phantom eye 210 arrangements. The reference image of each of the plurality of different phantom eyes 210 can be stored in a database associated with the particular phantom eye structure that generated each of the plurality of the reference images. A user can then select a biological eye of interest to study and determine the physiology, structure, and optic properties of the biological eye. Based on the determined physiology, structure, and optic properties of the biological eye, the database can be analyzed to determine the properties of the phantom eye 210 imaged that most closely mimic that of the biological eye. The reference image gathered of the phantom eye 210 that most closely mimics the biological eye of interest can then be selected in the database and used to correct the spectral image of the biological eye in the step 1514 of the method 1500.

It should be appreciated that the various steps 1502-1514 of the method 1500 can be carried out in any desirable order besides the order depicted in FIG. 17. For instance, in some embodiments, the position of one or more ocular media 250 components can be adjusted prior to adjusting the position and/or shape of the reflectance standard 230. Moreover, in some embodiments, the light source 202 can be instructed to illuminate the phantom eye 210 and the light source 202 can be adjusted according to the data from the one or more additional sensors 1206 (FIG. 14) prior to the position or shape of the one or more ocular media 250 components or the reflectance standard 230 being adjusted. It should, also, be appreciated that one or more of the steps 1502-1514 of the method 1500 can be carried out substantially simultaneously. For instance, in some embodiments, the position of one or more ocular media 250 components and the position or shape of the reflectance standard 230 can be adjusted substantially simultaneously.

The above-described phantom eyes, including curved reflectance standards and ocular media mimicking the optics of a biological eye of interest, have been shown to accurately correct or calibrate spectral images of a biological eye of interest.

### Benefits of Phantom Eye Mimicking Optical Power of Biological Eye

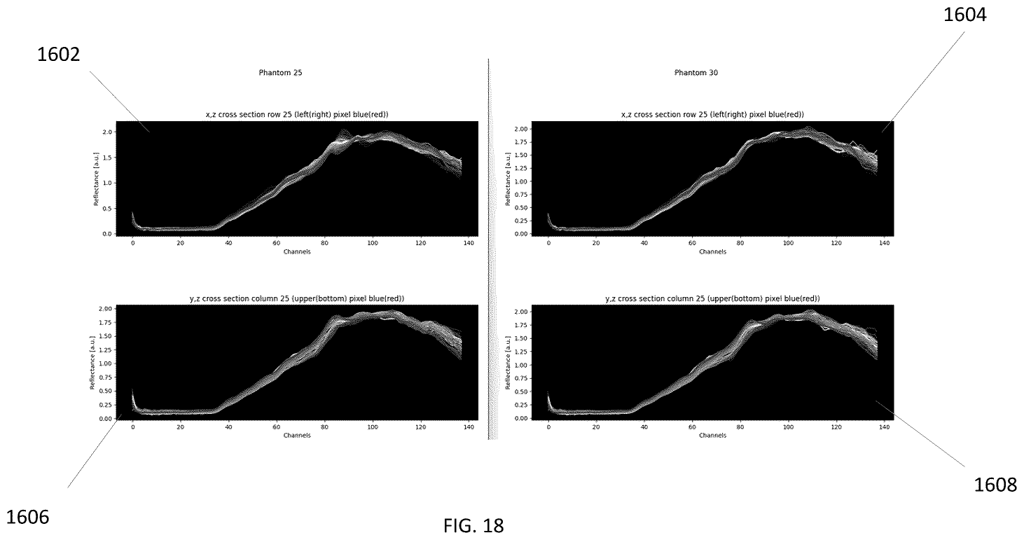

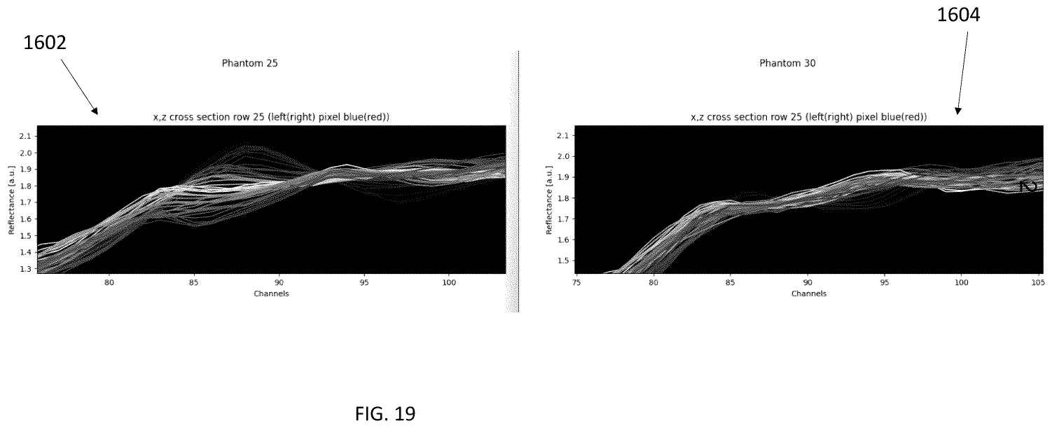

Referring now to FIG. 18, spectral curves are presented. Panels 1602 and 1604 present 50 single pixels spectral curves of row 25 on a matrix sensor of 50×50 pixels. Panels 1606 and 1608 present 50 single pixels spectral curves of column 25 on a matrix sensor of 50×50 pixels. Panels 1602 and 1606 represent the spectra of a fovea image from a hyperopic human eye, normalized using a myopic phantom eye (Phantom 25). Panels 1604 and 1608 represent the spectra of a fovea image from a hyperopic human eye, normalized using an emmetropic phantom eye (Phantom 30). Therefore, as discussed in detail above, the very same fovea acquisition was divided by two images of phantom eyes that have different total refractive powers. The horizontal axes of the panels 1602-1608 represent the spectral channels numbers associated with precise wavelength ranges of the light. The vertical axes of the panels 1602-1608 represent the normalized reflectance value of the retina.

Referring now to FIG. 19, magnified images of the panel 1602 and the panel 1604 are depicted. As can be noticed, the spectral curves of the Phantom 25-normalized image (the myopic phantom eye) show a considerable dependency on the selected pixel over the row. The Phantom 30-normalized image (emmetropic phantom eye) shows a smoother response and more consistency among different pixels, as it is expected by a reference. Therefore, the emmetropic phantom eye introduces less (or null) spectral dependency associated to the selected pixel on the sensor. This means that refractive power (retinal distance from the lens, lens power, ocular media, etc.) plays a role in the reference standard normalization, and for proper normalization, a phantom eye that is mimicking or closely approximating the refractive power and accommodation of the biological eye under study should be used.

### Benefits of Curved Reflectance Standard

Because some optical designs of retinal imagers are based on the assumption that the imaged object surface is curved, suboptimal imaging and spectral calibration is obtained when this assumption is “violated” when a flat, white reflectance standard surface is imaged. Some phantom eyes described herein, including curved reflectance standards, may correct for brightness and spectral and spatial aberrations that would result from the use of flat, white reflectance standard surfaces.

In some illumination schemes for fundus imaging using a flat, white reflectance standard, one can recognize an uneven illumination pattern or, worse, a “donut” on the white reflectance standard. The “donut” pattern is a brightness artifact generated by the illumination distribution of the light source on a flat surface, such as a flat reflectance standard. In a fundus image, the illumination distribution is more even and does not present abrupt discontinuities, as it is designed to wet a curved surface evenly. In practice, when an acquired fundus image, with an even illumination, is then divided by an image of this flat, white reflectance standard with a different illumination pattern on it, an artifact is introduced to the obtained spectral reflectance image. The phantom eyes described herein with curved reflectance standards may eliminate or mitigate the effects of this artifact, as the curvature is as close as possible to the one of a real retina and the lighting is designed to be as diffuse as possible on the curved reflectance standard. Moreover a curved reflectance standard is nearer to the optical design specification and introduce less optical aberrations, distortions and artifacts than a flat reference standard.

The acquired hyperspectral datacube can be oriented with a three axes system of x, y, and z coordinates. X and y coordinates represent the spatial location while the z coordinate identifies the spectral channels. The data can be presented as slice fixing one of the three coordinates and plot as an image the resulting rectangle.

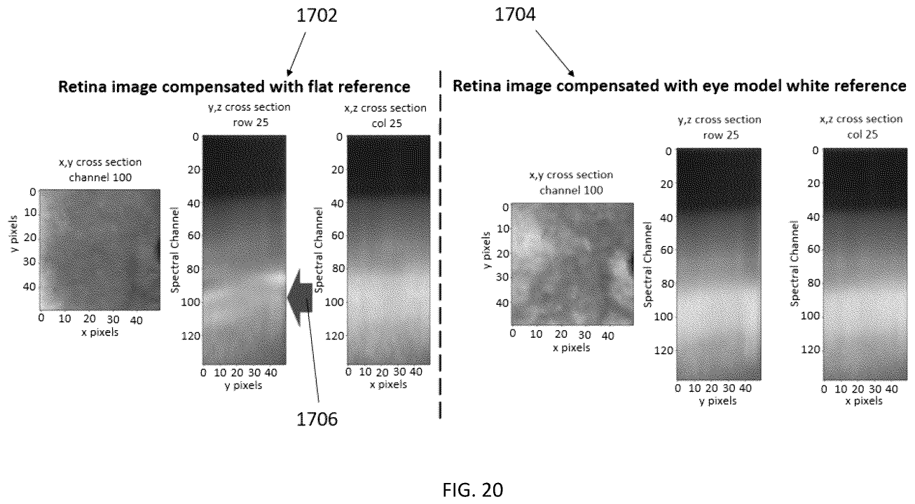

With respect to FIG. 20, two series of panels are presented. The series of panels 1702 on the left presents cross sections of a retinal hyperspectral datacube normalized with a flat, white reflectance standard. The series of panels 1704 on the right presents cross sections of a retinal hyperspectral datacube normalized with a phantom eye including a curved reflectance standard. For each single series of panels, from left to right, the plots presented are the x, y spatial cross section acquired at spectral channel 80, the y, z spectral cross section at row (y) 25, and the x, z cross section at column (x) 25. The arrow 1706 indicates the region where most of the optical aberrations of the white, flat reflectance standard distorts the spectral curves of the resultant retinal reflectance. In the cross sections x and y coordinates are pixel location on the sensor, and z coordinates are the spectral channel.

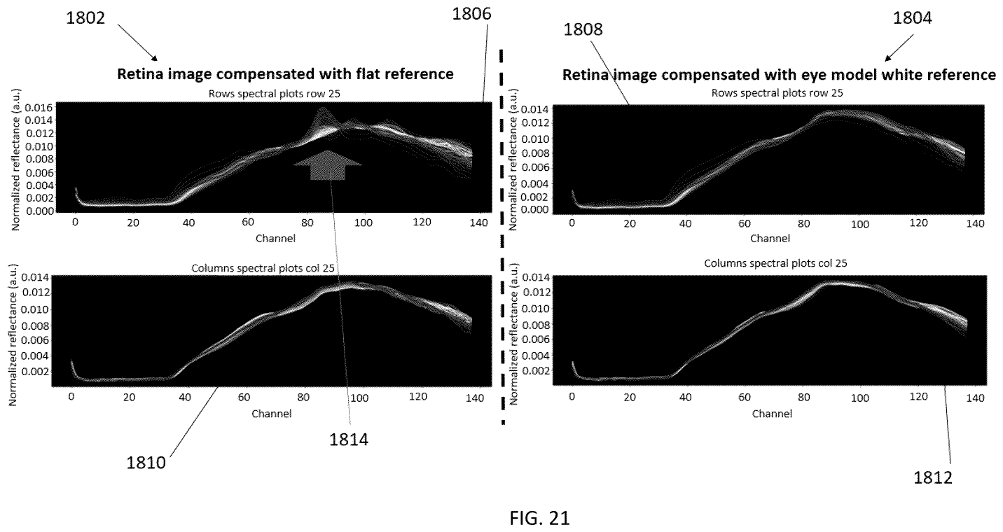

Referring now to FIG. 21, two series of panels, 1802 and 1804, are presented. The series of panels 1802 on the left presents spectral curves of a retinal hyperspectral datacube normalized with a flat, white reflectance standard. The series of panels 1804 on the right presents spectral curves of a retinal hyperspectral datacube normalized with a phantom eye including a curved reflectance standard. For each series of panels, the top panel (i.e. panels 1806 and 1808) present 50 single pixels spectral curves of row 25 on a matrix sensor of 50×50 pixels, and the bottom panels (i.e. panels 1810 and 1812) present 50 single pixels spectral curves of column 25 on a matrix sensor of 50×50 pixels. The arrow 1814 indicates the region where most of the optical aberrations of the white, flat reflectance standard distorts the spectral curves of the resultant retinal reflectance. In the plots, x and y coordinates are pixel location on the sensor, and z coordinates are the spectral channel.

As can be noticed from FIGS. 20 and 21, the flat, white reflectance standard normalization produces less smooth and less consistent spectral curves and introduces some spectral and spatial dependent artifacts in the reflectance curves. These inconsistencies are given by two effects. First, a flat, white reflectance standard target may be placed very near to the imaging system to cover the entire FOV, and that means that to be in focus, the refraction correction should be very high and therefore very distant to emmetropic nominal operating conditions. That means that the refraction correction with a flat reflectance standard is the worst possible, unless in the very unlikely case of an extremely myopic biological eye under study. The second effect is related to the optical aberrations and the fact that the curvature of the retina is not compensated (field curvature). The spectral curves for the white, flat reflectance standards are more dispersed both in reflectance values and in spectral channels. In fact, a clear shift can be recognized over the entire spectral range over the FOV (blue pixels (bottom pixels) are more left shifted than red pixels). That shift is bigger and makes the curves more dispersed in the case of a white, flat reflectance standard normalized retinal image. The spectral curves of the phantom eyes including curved reflectance standards, are far more compact and less affected by these differences in spectral information, as expected from a reference eye model.

Again, it can be concluded that a phantom eye mimicking the biological eye in all its components, including a curved reflectance standard, will give improved data quality compared to retinal image correction with a flat, white reference standard.

No-limiting embodiments of the present disclosure are set out in the following clauses:

1. A phantom eye, comprising: a curved reflectance standard having a light-receiving surface configured to be illuminated by light; and one or more ocular media components positioned such that the light passes through the one or more ocular media components prior to illuminating the light-receiving surface of the curved reflectance standard, wherein: the curved reflectance standard mimics an optical property of a retina of a biological eye; and the one or more ocular media components mimic an optical property of the biological eye.

2. The phantom eye of the preceding clause, wherein the curved reflectance standard comprises a material with a known reflectance.

3. The phantom eye of any preceding clause, further comprising a shell, wherein the shell at least partially defines an internal volume of the phantom eye.

4. The phantom eye of any preceding clause, wherein the curved reflectance standard and the shell at least partially define the internal volume of the phantom eye.

5. The phantom eye of any preceding clause, wherein the curved reflectance standard is secured to an inner surface of the shell.

6. The phantom eye of any preceding clause, wherein the shell comprises one or more receptors configured to removably mate with and retain the curved reflectance standard in the shell.

7. The phantom eye of any preceding clause, wherein the curved reflectance standard is coated on an inner surface of the shell.

8. The phantom eye of any preceding clause, wherein: the shell comprises a plurality of slots; each of the plurality of slots are positioned a different distance from the curved reflectance standard; and each of the plurality of slots are configured to receive at least one of the one or more ocular media components.

9. The phantom eye of any preceding clause, wherein the shell comprises an integrating sphere.

10. The phantom eye of any preceding clause, wherein the curved reflectance standard is coated on a surface of the integrating sphere.

11. The phantom eye of any preceding clause, wherein at least one of the one or more ocular media components and the shell at least partially define the internal volume of the phantom eye.

12. The phantom eye of any preceding clause, wherein at least one of the one or more ocular media components is secured to an inner surface of the shell.

13. The phantom eye of any preceding clause, wherein at least one of the one or more ocular media components is secured to an outer surface of the shell.

14. The phantom eye of any preceding clause, wherein the one or more ocular media components comprise an artificial sclera.

15. The phantom eye of any preceding clause, wherein the artificial sclera at least partially defines a shell of the phantom eye, the shell at least partially defining an internal volume of the phantom eye.

16. The phantom eye of any preceding clause, wherein the artificial sclera comprises at least one of Polycaprolactone (PCL), glass, or Polymethyl-methacrylate (PMMA).

17. The phantom eye of any preceding clause, wherein the artificial sclera is configured to mimic an optical property of a biological sclera of the biological eye.

18. The phantom eye of any preceding clause, wherein the one or more ocular media components comprise an artificial cornea.

19. The phantom eye of any preceding clause, wherein the artificial cornea comprises at least one of polydimethylsiloxane (PDMS), glass, or Polymethyl-methacrylate (PMMA).

20. The phantom eye of any preceding clause, wherein the artificial cornea is configured to mimic an optical property of a biological cornea of the biological eye.

21. The phantom eye of any preceding clause, wherein the one or more ocular media components comprise an artificial iris.

22. The phantom eye of any preceding clause, wherein the artificial iris comprises a fluidic system based on electrowetted-actuated mixtures of materials.

23. The phantom eye of any preceding clause, wherein the artificial iris comprises a tunable diaphragm.

24. The phantom eye of any preceding clause, wherein the artificial iris is configured to mimic an optical property of a biological iris of the biological eye.

25. The phantom eye of any preceding clause, wherein the one or more ocular media components comprise an artificial crystalline lens.

26. The phantom eye of any preceding clause, wherein the artificial crystalline lens comprises at least one of polydimethylsiloxane (PDMS), glass, or Polymethyl-methacrylate (PMMA).

27. The phantom eye of any preceding clause, wherein the artificial crystalline lens comprises at least one of a plano-concave lens, a pano-convex lens, a meniscus lens, a double convex lens, or a double concave lens.

28. The phantom eye of any preceding clause, wherein the artificial crystalline lens is configured to mimic an optical property of a biological crystalline lens of the biological eye.

29. The phantom eye of any preceding clause, further comprising a shell, wherein: the shell at least partially defines an internal volume of the phantom eye; the curved reflectance standard, the shell, and the artificial crystalline lens at least partially define a cavity therebetween; the one or more ocular media components comprise an artificial vitreous humor; and the artificial vitreous humor is positioned in the cavity.

30. The phantom eye of any preceding clause, wherein the artificial vitreous humor is configured to mimic an optical property of a biological vitreous humor of the biological eye.

31. The phantom eye of any preceding clause, wherein the one or more ocular media components comprise: an artificial crystalline lens; and an artificial cornea, wherein the artificial cornea is positioned anterior of the artificial crystalline lens.

32. The phantom eye of any preceding clause, wherein the one or more ocular media components comprise an artificial iris, wherein: the artificial iris is positioned anterior of the artificial crystalline lens; and the artificial iris is positioned posterior of the artificial cornea.

33. The phantom eye of any preceding clause, wherein the one or more ocular media components comprise an artificial aqueous humor, wherein: the artificial crystalline lens, the artificial cornea, and the artificial iris at least partially define a cavity therebetween; and the artificial aqueous humor is positioned in the cavity.

34. The phantom eye of any preceding clause, wherein the artificial aqueous humor is configured to mimic an optical property of a biological aqueous humor of the biological eye.

35. The phantom eye of any preceding clause, further comprising a spectral filter.

36. The phantom eye of any preceding clause, further comprising an anterior surface, wherein the spectral filter is positioned on the anterior surface of the phantom eye.

37. The phantom eye of any preceding clause, further comprising an enclosure, wherein the curved reflectance standard and at least one of the one or more ocular media components are housed within the enclosure.

38. The phantom eye of any preceding clause, wherein: the enclosure comprises a curved cutout; and the curved reflectance standard and at least one of the one or more ocular media components are positioned within the curved cutout.

39. The phantom eye of any preceding clause, wherein: the curved reflectance standard is positioned against a surface of the curved cutout; and at least one of the one or more ocular media components are positioned against an anterior surface of the curved reflectance standard.

40. The phantom eye of any preceding clause, further comprising an actuator, wherein: the actuator is positioned within the enclosure; and the actuator is coupled to the curved reflectance standard and configured to move the curved reflectance standard.

41. The phantom eye of any preceding clause, further comprising an actuator, wherein: at least one of the one or more ocular media components is coupled to the curved reflectance standard; and the actuator is configured to move the curved reflectance standard and the at least one of the one or more ocular media components coupled to the curved reflectance standard.

42. The phantom eye of any preceding clause, wherein the enclosure comprises a mounting member configured to mount the enclosure to an apparatus configured to move the enclosure.

43. The phantom eye of any preceding clause, wherein the mounting member is a cavity in an outer surface of the enclosure.

44. The phantom eye of any preceding clause, wherein the mounting member is a protrusion extending from an outer surface of the enclosure.

45. The phantom eye of any preceding clause, wherein the curved reflectance standard is interchangeable with another curved reflectance standard.

46. The phantom eye of any preceding clause, wherein the one or more ocular media components are interchangeable with one or more other ocular media components.

47. The phantom eye of any preceding clause, wherein at least one of the one or more ocular media components is coupled to a movable track, wherein the movable track is configured to adjust a distance between the at least one of the one or more ocular media components and the curved reflectance standard.

48. The phantom eye of any preceding clause, wherein the curved reflectance standard is coupled to a movable track, wherein the movable track is configured to adjust a distance between the one or more ocular media components and the curved reflectance standard.

49. The phantom eye of any preceding clause, wherein at least one of the one or more ocular media components is coupled to a rotary wheel, and wherein the rotary wheel is configured to selectively position the at least one of the one or more ocular media components in anterior of the curved reflectance standard.

50. The phantom eye of any preceding clause, further comprising a selectively deformable component coupled to at least one of the curved reflectance standard or at least one of the one or more ocular media components, wherein the selectively deformable component is configured to adjust a curvature of the curved reflectance standard or the at least one of the one or more ocular media components.

51. The phantom eye of any preceding clause, wherein the optical property of the biological eye comprises at least one of a refractive power or a transmittance.

52. The phantom eye of any preceding clause, wherein the optical property of the retina comprises a curvature.

53. A system, comprising: a phantom eye, comprising: a curved reflectance standard having a light-receiving surface configured to be illuminated by light; and one or more ocular media components positioned such that the light passes through the one or more ocular media components prior to illuminating the light-receiving surface of the curved reflectance standard, wherein: the curved reflectance standard mimics an optical property of a retina of a biological eye; and the one or more ocular media components mimic an optical property of the biological eye; a light source configured to emit light to the curved reflectance standard; and a sensor configured to detect light reflected by the curved reflectance standard.

54. The system of the preceding clause, wherein the one or more ocular media components are positioned between the light source and the curved reflectance standard along an optical path of the light emitted from the light source to the curved reflectance standard.

55. The system of any preceding clause, wherein the one or more ocular media components are positioned between the curved reflectance standard and the sensor along an optical path of light reflected to the sensor from the curved reflectance standard.

56. The system of any preceding clause, wherein the one or more ocular media components are positioned: between the light source and the curved reflectance standard along an optical path of the light emitted from the light source to the curved reflectance standard; and between the curved reflectance standard and the sensor along an optical path of light reflected to the sensor from the curved reflectance standard.

57. The system of any preceding clause, wherein the one or more ocular media components comprise an artificial sclera configured to mimic an optical property of a biological sclera of the biological eye.

58. The system of any preceding clause, wherein the one or more ocular media components comprise an artificial cornea configured to mimic an optical property of a biological cornea of the biological eye.

59. The system of any preceding clause, wherein the one or more ocular media components comprise an artificial iris configured to mimic an optical property of a biological iris of the biological eye.

60. The system of any preceding clause, wherein the one or more ocular media components comprise an artificial crystalline lens configured to mimic an optical property of a biological crystalline lens of the biological eye.

61. The system of any preceding clause, wherein the one or more ocular media components comprise an artificial vitreous humor configured to mimic an optical property of a biological vitreous humor of the biological eye.

62. The system of any preceding clause, wherein the one or more ocular media components comprise an artificial aqueous humor configured to mimic an optical property of a biological aqueous humor of the biological eye.

63. The system of any preceding clause, further comprising a spectral filter, wherein the spectral filter is positioned between the light source and the curved reflectance standard along an optical path of the light emitted from the light source to the curved reflectance standard.

64. The system of any preceding clause, wherein the spectral filter is positioned between the light source and the one or more ocular media components along an optical path of the light emitted from the light source to the curved reflectance standard.

65. The system of any preceding clause, further comprising a spectral filter, wherein the spectral filter is positioned between the curved reflectance standard and the sensor along an optical path of light reflected to the sensor from the curved reflectance standard.

66. The system of any preceding clause, wherein the spectral filter is positioned between the one or more ocular media components and the sensor along an optical path of light reflected to the sensor from the curved reflectance standard.

67. The system of any preceding clause, further comprising a spectral filter, wherein the spectral filter is positioned: between the light source and the curved reflectance standard along an optical path of the light emitted from the light source to the curved reflectance standard; and between the curved reflectance standard and the sensor along an optical path of light reflected to the sensor from the curved reflectance standard.

68. The system of any preceding clause, further comprising an apparatus comprising an actuator, wherein: the phantom eye further comprises an enclosure; the curved reflectance standard and at least one of the one or more ocular media components are housed within the enclosure; the enclosure comprises a mounting member configured to couple the enclosure to the apparatus; and the actuator is configured to adjust a position of the phantom eye.

69. The system of any preceding clause, further comprising a movable track, wherein: at least one of the one or more ocular media components is coupled to the movable track; and the movable track is configured to move the at least one of the one or more ocular media components to adjust a distance between the at least one of the one or more ocular media components and the curved reflectance standard.

70. The system of any preceding clause, further comprising a movable track, wherein: the curved reflectance standard is coupled to a movable track; and the movable track is configured to move the curved reflectance standard to adjust a distance between the one or more ocular media components and the curved reflectance standard.

71. The system of any preceding clause, further comprising a rotary wheel, wherein: at least one of the one or more ocular media components is coupled to a rotary wheel; and the rotary wheel is configured to selectively position the at least one of the one or more ocular media components in at least one of the following positions: between the light source and the curved reflectance standard along an optical path of the light emitted from the light source to the curved reflectance standard; or between the curved reflectance standard and the sensor along an optical path of light reflected to the sensor from the curved reflectance standard.

72. The system of any preceding clause, further comprising a beam splitter, wherein: the beam splitter is positioned between the light source and the phantom eye along an optical path of light emitted by the light source to the curved reflectance standard; and the beam splitter is configured to re-direct a portion of incoming irradiance along the optical path as a split beam.

73. The system of any preceding clause, further comprising one or more fiber end holders and one or more optical sensors, wherein each of the one or more fiber end holders is configured to couple a respective optical fiber of a respective optical sensor of the one or more optical sensors with the split beam.

74. The system of any preceding clause, wherein the phantom eye further comprises an integrating sphere at least partially defining an internal volume of the phantom eye, and wherein the curved reflectance standard is positioned within an interior of the integrating sphere.

75. The system of any preceding clause, wherein the system further comprises a second sensor, and the integrating sphere comprises: a first opening to allow light emitted from the light source to the curved reflectance standard into the integrating sphere and to allow light reflected by the curved reflectance standard to the sensor out of the integrating sphere; and a second opening, wherein the second sensor has a sensing area directed toward the interior of the integrating sphere through the second opening and is configured to measure properties of the light reflected by the curved reflectance standard.

76. The system of any preceding clause, wherein: the integrating sphere comprises a baffle in the interior of the integrating sphere; and the baffle is configured to block the second sensor from light emitted from the light source.

77. A method of using a phantom eye, comprising: imaging a phantom eye with a light source of a lighting assembly to generate a reference image, wherein the phantom eye comprises: a curved reflectance standard having a light-receiving surface configured to be illuminated by light; and one or more ocular media components positioned between the curved reflectance standard and the lighting assembly, wherein: the curved reflectance standard mimics an optical property of a retina of a biological eye; and the one or more ocular media components mimic an optical property of the biological eye; imaging the biological eye with the light source of the lighting assembly to generate a spectral image of the biological eye; and adjusting the spectral image of the biological eye based at least in part on the reference image.

78. The method of the preceding clause, further comprising moving the phantom eye relative the lighting assembly prior to imaging the phantom eye.

79. The method of any preceding clause, wherein moving the phantom eye comprises moving the phantom eye via one or more actuators coupled to the phantom eye.

80. The method of any preceding clause, further comprising adjusting a position of the curved reflectance standard within the phantom eye prior to imaging the phantom eye.

81. The method of any preceding clause, wherein adjusting the position of the curved reflectance standard comprises adjusting the position of the curved reflectance standard via one or more actuators coupled to the curved reflectance standard.

82. The method of any preceding clause, wherein the one or more actuators comprises a movable track.

83. The method of any preceding clause, wherein adjusting the position of the curved reflectance standard comprises changing the position of the curved reflectance standard relative the one or more ocular media components to mimic the optical properties of the biological eye.

84. The method of any preceding clause, further comprising adjusting a curvature of the curved reflectance standard within the phantom eye prior to imaging the phantom eye.

85. The method of any preceding clause, wherein adjusting the curvature of the curved reflectance standard comprises adjusting the curvature of the curved reflectance standard via a selectively deformable component coupled to the curved reflectance standard.

86. The method of any preceding clause, wherein the curvature of the curved reflectance standard is adjusted to mimic a curvature of the retina of the biological eye.

87. The method of any preceding clause, further comprising interchanging a first curved reflectance standard with a second curved reflectance standard prior to imaging the phantom eye, wherein the second curved reflectance standard mimics the optical property of the retina of the biological eye.

88. The method of any preceding clause, wherein the first curved reflectance standard and the second curved reflectance standard are removably attachable to the phantom eye.

89. The method of any preceding clause, further comprising adjusting a position of at least one of the one or more ocular media components prior to imaging the phantom eye.

90. The method of any preceding clause, wherein adjusting the position of the at least one of the one or more ocular media components comprises adjusting the position of the at least one of the one or more ocular media components via one or more actuators coupled to the at least one of the one or more ocular media components.

91. The method of any preceding clause, wherein the one or more actuators comprises a movable track.

92. The method of any preceding clause, wherein adjusting the position of the at least one of the one or more ocular media components comprises selectively positioning the at least one of the one or more ocular media components in one of a plurality of slots of the phantom eye.

93. The method of any preceding clause, wherein adjusting the position of the at least one of the one or more ocular media components comprises changing the position of the at least one of the one or more ocular media components relative the curved reflectance standard to mimic the optical property of the biological eye.

94. The method of any preceding clause, further comprising interchanging a first ocular media component with a second ocular media component prior to imaging the phantom eye, wherein the second ocular media component mimics the optical property of the biological eye.

95. The method of any preceding clause, wherein the first ocular media component and the second ocular media component are removably attachable to the phantom eye.

96. The method of any preceding clause, wherein the first ocular media component and the second ocular media component are coupled to a rotary wheel, and wherein interchanging the first ocular media component with the second ocular media component comprises rotating the rotary wheel to position the second ocular media component between the curved reflectance standard and the lighting assembly.

97. The method of any preceding clause, further comprising adjusting a curvature of at least one of the one or more ocular media components prior to imaging the phantom eye.

98. The method of any preceding clause, wherein adjusting the curvature of the at least one of the one or more ocular media components comprises adjusting the curvature of the at least one of the one or more ocular media components via a selectively deformable component coupled to the curved reflectance standard.

99. The method of any preceding clause, wherein the curvature of the at least one of the one or more ocular media components is adjusted to mimic a curvature of a component of the biological eye.

100. The method of any preceding clause, wherein imaging the phantom eye with the light source of the lighting assembly comprises: determining one or more properties of light emitted from the light source to the curved reflectance standard; and adjusting one or more parameters of the light source based on the one or more properties.

101. The method of any preceding clause, wherein the one or more properties are determined with one or more light sensors.

102. The method of any preceding clause, wherein imaging the phantom eye with the light source of the lighting assembly to generate the reference image comprises: directing light from the light source to the curved reflectance standard; and collecting light reflected by the curved reflectance standard with a sensor.

103. The method of any preceding clause, wherein the reference image is a spectral image of the curved reflectance standard.

104. The method of any preceding clause, wherein adjusting the spectral image of the biological eye based at least in part on the reference image comprises dividing the spectral image of the biological eye by the reference image.

105. A system, comprising: a phantom eye; a lighting assembly, comprising: a light source configured to direct light to the phantom eye; and a sensor configured to detect light reflected from the phantom eye; and a processor in communication with the lighting assembly and programmed to correct a spectral retinal image with a spectral calibration image of the phantom eye.

106. The system of the preceding clause, further comprising an actuator coupled to the phantom eye, wherein: the processor is in communication with the actuator; and the processor is programmed to drive the actuator to move the phantom eye relative the lighting assembly.

107. The system of any preceding clause, further comprising an actuator, wherein: the phantom eye comprises a curved reflectance standard and one or more ocular media components positioned between the curved reflectance standard and the lighting assembly; the actuator is coupled to the curved reflectance standard; the processor is in communication with the actuator; and the processor is programmed to drive the actuator to move the curved reflectance standard relative the one or more ocular media components.

108. The system of any preceding clause, wherein the actuator is a movable track.

109. The system of any preceding clause, further comprising a selectively deformable component, wherein: the phantom eye comprises a curved reflectance standard; the selectively deformable component is coupled to the curved reflectance standard; the processor is in communication with the selectively deformable component; and the processor is programmed to deform the selectively deformable component to adjust a curvature of the curved reflectance standard.

110. The system of any preceding clause, further comprising an actuator, wherein: the phantom eye comprises a curved reflectance standard and one or more ocular media components positioned between the curved reflectance standard and the lighting assembly; the actuator is coupled to at least one of the one or more ocular media components; the processor is in communication with the actuator; and the processor is programmed to drive the actuator to move the one or more ocular media components relative the curved reflectance standard.

111. The system of any preceding clause, wherein the actuator is a movable track.

112. The system of any preceding clause, further comprising a rotary wheel, wherein: the phantom eye comprises: a curved reflectance standard; a first ocular media component; and a second ocular media component; the first ocular media component and the second ocular media component are coupled to the rotary wheel; the processor is in communication with the rotary wheel; and the processor is programmed to rotate the rotary wheel to position one of the first ocular media component or the second ocular media component between the curved reflectance standard and the lighting assembly.

113. The system of any preceding clause, further comprising a selectively deformable component, wherein: the phantom eye comprises one or more ocular media components; the selectively deformable component is coupled to at least one of the one or more ocular media components; the processor is in communication with the selectively deformable component; and the processor is programmed to deform the selectively deformable component to adjust a curvature of the at least one of the one or more ocular media components.

114. The system of any preceding clause, further comprising an additional light sensor, wherein: the processor is in communication with additional light sensor; the additional light sensor is configured to detect one or more properties of the light directed from the light source to the phantom eye; and the processor is programmed to adjust one or more parameters of the light source based on the one or more properties.

115. The system of any preceding clause, further comprising a beam splitter, wherein: the beam splitter is positioned between the lighting assembly and the phantom eye; the beam splitter is configured to re-direct a portion of incoming irradiance as a split beam; and the additional light sensor is configured to detect one or more properties of the split beam.

116. The system of any preceding clause, further comprising an additional light sensor, wherein: the processor is in communication with additional light sensor; the additional light sensor is configured to detect one or more properties of the light reflected from the phantom eye; and the processor is programmed to determine a functionality of the phantom eye based on the one or more properties.

117. The system of any preceding clause, wherein correcting the spectral retinal image with the spectral calibration image of the phantom eye comprises dividing the spectral retinal image by the spectral calibration image of the phantom eye.

118. The system of any preceding clause, wherein the processor is programmed to adjust one or more components of the phantom eye such that the phantom eye approximates the optical properties of a biological eye featured in the spectral retinal image.

119. The system of any preceding clause, wherein the processor is programmed to: access a database of spectral calibration images of a plurality of phantom eyes, wherein each spectral calibration image is associated with a respective phantom eye of unique optical properties that the spectral calibration image was generated from; identify a first spectral calibration image, wherein the first spectral calibration image is associated with a first phantom eye that mimics optical properties of a biological eye that the spectral retinal image was generated from; and correct the spectral retinal image with the first spectral calibration image of the first phantom eye.

120. An apparatus comprising: at least one processor; and at least one storage medium having encoded thereon executable instructions that, when executed by the at least one processor, cause the at least one processor to carry out a method comprising: adjusting a hyperspectral image of a biological eye based at least in part on a reference image captured using a phantom eye, the phantom eye comprising: a curved reflectance standard having a light-receiving surface configured to be illuminated by light; and one or more ocular media components positioned such that the light passes through the one or more ocular media components prior to illuminating the light-receiving surface of the curved reflectance standard.

121. A method of using a phantom eye, comprising: imaging a phantom eye with a light source of a lighting assembly to generate a reference image, wherein the reference image is configured to be used to calibrate an imaging system, and wherein the phantom eye comprises: a curved reflectance standard having a light-receiving surface configured to be illuminated by light; and one or more ocular media components positioned between the curved reflectance standard and the lighting assembly, wherein: the curved reflectance standard mimics an optical property of a retina of a biological eye; and the one or more ocular media components mimic an optical property of the biological eye.

122. The method of the preceding clause, further comprising moving the phantom eye relative the lighting assembly prior to imaging the phantom eye.

123. The method of any preceding clause, wherein moving the phantom eye comprises moving the phantom eye via one or more actuators coupled to the phantom eye.

124. The method of any preceding clause, further comprising adjusting a position of the curved reflectance standard within the phantom eye prior to imaging the phantom eye.

125. The method of any preceding clause, wherein adjusting the position of the curved reflectance standard comprises adjusting the position of the curved reflectance standard via one or more actuators coupled to the curved reflectance standard.

126. The method of any preceding clause, wherein the one or more actuators comprises a movable track.

127. The method of any preceding clause, wherein adjusting the position of the curved reflectance standard comprises changing the position of the curved reflectance standard relative the one or more ocular media components to mimic the optical properties of the biological eye.

128. The method of any preceding clause, further comprising adjusting a curvature of the curved reflectance standard within the phantom eye prior to imaging the phantom eye.

129. The method of any preceding clause, wherein adjusting the curvature of the curved reflectance standard comprises adjusting the curvature of the curved reflectance standard via a selectively deformable component coupled to the curved reflectance standard.

130. The method of any preceding clause, wherein the curvature of the curved reflectance standard is adjusted to mimic a curvature of the retina of the biological eye.

131. The method of any preceding clause, further comprising interchanging a first curved reflectance standard with a second curved reflectance standard prior to imaging the phantom eye, wherein the second curved reflectance standard mimics the optical property of the retina of the biological eye.

132. The method of any preceding clause, wherein the first curved reflectance standard and the second curved reflectance standard are removably attachable to the phantom eye.

133. The method of any preceding clause, further comprising adjusting a position of at least one of the one or more ocular media components prior to imaging the phantom eye.

134. The method of any preceding clause, wherein adjusting the position of the at least one of the one or more ocular media components comprises adjusting the position of the at least one of the one or more ocular media components via one or more actuators coupled to the at least one of the one or more ocular media components.

135. The method of any preceding clause, wherein the one or more actuators comprises a movable track.

136. The method of any preceding clause, wherein adjusting the position of the at least one of the one or more ocular media components comprises selectively positioning the at least one of the one or more ocular media components in one of a plurality of slots of the phantom eye.

137. The method of any preceding clause, wherein adjusting the position of the at least one of the one or more ocular media components comprises changing the position of the at least one of the one or more ocular media components relative the curved reflectance standard to mimic the optical property of the biological eye.

138. The method of any preceding clause, further comprising interchanging a first ocular media component with a second ocular media component prior to imaging the phantom eye, wherein the second ocular media component mimics the optical property of the biological eye.

139. The method of any preceding clause, wherein the first ocular media component and the second ocular media component are removably attachable to the phantom eye.

140. The method of any preceding clause, wherein the first ocular media component and the second ocular media component are coupled to a rotary wheel, and wherein interchanging the first ocular media component with the second ocular media component comprises rotating the rotary wheel to position the second ocular media component between the curved reflectance standard and the lighting assembly.

141. The method of any preceding clause, further comprising adjusting a curvature of at least one of the one or more ocular media components prior to imaging the phantom eye.

142. The method of any preceding clause, wherein adjusting the curvature of the at least one of the one or more ocular media components comprises adjusting the curvature of the at least one of the one or more ocular media components via a selectively deformable component coupled to the curved reflectance standard.

143. The method of any preceding clause, wherein the curvature of the at least one of the one or more ocular media components is adjusted to mimic a curvature of a component of the biological eye.

144. The method of any preceding clause, wherein imaging the phantom eye with the light source of the lighting assembly comprises: determining one or more properties of light emitted from the light source to the curved reflectance standard; and adjusting one or more parameters of the light source based on the one or more properties.

145. The method of any preceding clause, wherein the one or more properties are determined with one or more light sensors.

146. The method of any preceding clause, wherein imaging the phantom eye with the light source of the lighting assembly to generate the reference image comprises: directing light from the light source to the curved reflectance standard; and collecting light reflected by the curved reflectance standard with a sensor.

147. The method of any preceding clause, wherein the reference image is a spectral image of the curved reflectance standard.

148. At least one non-transitory computer-readable storage medium having encoded thereon executable instructions that, when executed by at least one processor, cause the at least one processor to carry out a method comprising: adjusting a hyperspectral image of a biological eye based at least in part on a reference image captured using a phantom eye, the phantom eye comprising: a curved reflectance standard having a light-receiving surface configured to be illuminated by light; and one or more ocular media components positioned such that the light passes through the one or more ocular media components prior to illuminating the light-receiving surface of the curved reflectance standard.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by the appended claims and the applicable rules of law.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one nonlimiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-148. (canceled)

149. A phantom eye, comprising:
a curved reflectance standard having a light-receiving surface configured to be illuminated by light; and
one or more ocular media components positioned such that the light passes through the one or more ocular media components prior to illuminating the light-receiving surface of the curved reflectance standard, wherein:
the curved reflectance standard mimics an optical property of a retina of a biological eye; and
the one or more ocular media components mimic an optical property of the biological eye.

150. The phantom eye of claim 149, further comprising a spectral filter positioned on an anterior surface of the phantom eye.

151. The phantom eye of claim 149, further comprising an enclosure, wherein the curved reflectance standard and at least one of the one or more ocular media components are housed within the enclosure.

152. The phantom eye of claim 151, wherein:
the enclosure comprises a curved cutout;
the curved reflectance standard and at least one of the one or more ocular media components are positioned within the curved cutout;
the curved reflectance standard is positioned against a surface of the curved cutout; and
at least one of the one or more ocular media components are positioned against an anterior surface of the curved reflectance standard.

153. The phantom eye of claim 151 further comprising an actuator, wherein:
the actuator is positioned within the enclosure; and
the actuator is coupled to the curved reflectance standard and configured to move the curved reflectance standard.

154. The phantom eye of claim 153, wherein:
at least one of the one or more ocular media components is coupled to the curved reflectance standard; and
the actuator is configured to move the curved reflectance standard and the at least one of the one or more ocular media components coupled to the curved reflectance standard.

155. The phantom eye of claim 151, wherein the enclosure comprises a mounting member configured to mount the enclosure to an apparatus configured to move the enclosure.

156. The phantom eye of claim 155, wherein the mounting member is a cavity in an outer surface of the enclosure or a protrusion extending from an outer surface of the enclosure.

157. The phantom eye of claim 149, wherein at least one of the one or more ocular media components is coupled to a movable track, wherein the movable track is configured to adjust a distance between the at least one of the one or more ocular media components and the curved reflectance standard.

158. The phantom eye of claim 149, wherein the curved reflectance standard is coupled to a movable track, wherein the movable track is configured to adjust a distance between the one or more ocular media components and the curved reflectance standard.

159. The phantom eye of claim 149, wherein at least one of the one or more ocular media components is coupled to a rotary wheel, and wherein the rotary wheel is configured to selectively position the at least one of the one or more ocular media components in anterior of the curved reflectance standard.

160. The phantom eye of claim 149, further comprising a selectively deformable component coupled to at least one of the curved reflectance standard or at least one of the one or more ocular media components, wherein the selectively deformable component is configured to adjust a curvature of the curved reflectance standard or the at least one of the one or more ocular media components.

161. A system, comprising:
a phantom eye, comprising:
a curved reflectance standard having a light-receiving surface configured to be illuminated by light; and
one or more ocular media components positioned such that the light passes through the one or more ocular media components prior to illuminating the light-receiving surface of the curved reflectance standard, wherein:
the curved reflectance standard mimics an optical property of a retina of a biological eye; and
the one or more ocular media components mimic an optical property of the biological eye;
a light source configured to emit light to the curved reflectance standard; and
a sensor configured to detect light reflected by the curved reflectance standard.

162. The system of claim 161, further comprising a movable track, wherein:
at least one of the one or more ocular media components or the curved reflectance standard is coupled to the movable track; and
the movable track is configured to move the at least one of the one or more ocular media components or the curved reflectance standard relative to the other to adjust a distance between the at least one of the one or more ocular media components and the curved reflectance standard.

163. A method of using a phantom eye, comprising:
imaging a phantom eye with a light source of a lighting assembly to generate a reference image, wherein the phantom eye comprises:
a curved reflectance standard having a light-receiving surface configured to be illuminated by light; and
one or more ocular media components positioned between the curved reflectance standard and the lighting assembly, wherein:
the curved reflectance standard mimics an optical property of a retina of a biological eye; and
the one or more ocular media components mimic an optical property of the biological eye;
imaging the biological eye with the light source of the lighting assembly to generate a spectral image of the biological eye; and
adjusting the spectral image of the biological eye based at least in part on the reference image.

164. The method of claim 163, further comprising moving the phantom eye via one or more actuators coupled to the phantom eye relative the lighting assembly prior to imaging the phantom eye.

165. The method of claim 163, further comprising adjusting a position of at least one of:
the curved reflectance standard within the phantom eye, via one or more actuators coupled to the curved reflectance standard, prior to imaging the phantom eye; or
at least one of the one or more ocular media components, via one or more actuators coupled to the at least one of the one or more ocular media components, prior to imaging the phantom eye.

166. A system, comprising:
a phantom eye;
a lighting assembly, comprising:
a light source configured to direct light to the phantom eye; and
a sensor configured to detect light reflected from the phantom eye; and
a processor in communication with the lighting assembly and programmed to correct a spectral retinal image with a spectral calibration image of the phantom eye.

167. The system of claim 166, further comprising an actuator, wherein:
the phantom eye comprises a curved reflectance standard and one or more ocular media components positioned between the curved reflectance standard and the lighting assembly;
the actuator is coupled to the curved reflectance standard or to at least one of the one or more ocular media components;
the processor is in communication with the actuator; and
the processor is programmed to drive the actuator to move the curved reflectance standard or the at least one of the one or more ocular media components relative the other.

168. The system of claim 166, further comprising a selectively deformable component, wherein:
the phantom eye comprises one or more ocular media components;
the selectively deformable component is coupled to at least one of the one or more ocular media components;
the processor is in communication with the selectively deformable component; and
the processor is programmed to deform the selectively deformable component to adjust a curvature of the at least one of the one or more ocular media components.

169. The system of claim 166, wherein the processor is programmed to adjust one or more components of the phantom eye such that the phantom eye approximates one or more optical properties of a biological eye featured in the spectral retinal image.

170. The system of claim 166, wherein the processor is programmed to:
access a database of spectral calibration images of a plurality of phantom eyes, wherein each spectral calibration image is associated with a respective phantom eye of unique optical properties that the spectral calibration image was generated from;
identify a first spectral calibration image, wherein the first spectral calibration image is associated with a first phantom eye that mimics optical properties of a biological eye that the spectral retinal image was generated from; and
correct the spectral retinal image with the first spectral calibration image of the first phantom eye.

171. An apparatus comprising:
at least one processor; and
at least one storage medium having encoded thereon executable instructions that, when executed by the at least one processor, cause the at least one processor to carry out a method comprising:
adjusting a hyperspectral image of a biological eye based at least in part on a reference image captured using a phantom eye, the phantom eye comprising:
a curved reflectance standard having a light-receiving surface configured to be illuminated by light; and
one or more ocular media components positioned such that the light passes through the one or more ocular media components prior to illuminating the light-receiving surface of the curved reflectance standard.

172. A method of using a phantom eye, comprising:
imaging a phantom eye with a light source of a lighting assembly to generate a reference image, wherein the reference image is configured to be used to calibrate an imaging system, and wherein the phantom eye comprises:
a curved reflectance standard having a light-receiving surface configured to be illuminated by light; and
one or more ocular media components positioned between the curved reflectance standard and the lighting assembly, wherein:
the curved reflectance standard mimics an optical property of a retina of a biological eye; and
the one or more ocular media components mimic an optical property of the biological eye.

173. At least one non-transitory computer-readable storage medium having encoded thereon executable instructions that, when executed by at least one processor, cause the at least one processor to carry out a method comprising:
adjusting a hyperspectral image of a biological eye based at least in part on a reference image captured using a phantom eye, the phantom eye comprising:
a curved reflectance standard having a light-receiving surface configured to be illuminated by light; and
one or more ocular media components positioned such that the light passes through the one or more ocular media components prior to illuminating the light-receiving surface of the curved reflectance standard.
